(19) United States
(12) Patent Application Publication
(10) Pub. No.: US 2026/0327268 A1
(43) Pub. Date: Oct. 22, 2026

(54) FITNESS MATURATION OF ENGINEERED AAV CAPSID STAC-102

(71) Applicant: Sangamo Therapeutics, Inc., Richmond, CA (US)

(72) Inventors: David S. Ojala, Richmond, CA (US); Matthew R. Tiffany, San Mateo, CA (US)

(73) Assignee: Sangamo Therapeutics, Inc., Richmond, CA (US)

(21) Appl. No.: 19/483,954

(22) PCT Filed: May 14, 2024
(86) PCT No.: PCT/US2024/029331

§ 371 Date: Nov. 12, 2025

Related U.S. Application Data
(60) Provisional application No. 63/466,579, filed on May 15, 2023.

(51) Int. Cl. C07K 14/005

(52) U.S. Cl. CPC C07K 14/005; C12N 2750/14122

(57) ABSTRACT

This application relates to engineering AAV capsids, for example, STAC-102. In some embodiments, the engineered AAV capsids mediate delivery to the central nervous system.

### CROSS REFERENCE TO RELATED APPLICATION

This application claims the benefit of U.S. Provisional Patent Application No. 63/466,579 filed on May 15, 2023 and entitled “Engineered AAV Capsids for CNS Targeting,” the entire contents of which are incorporated by reference herein.

### FIELD

This application relates to engineering AAV Capsids.

### BACKGROUND

The clinical translation of genomic medicines to treat disorders of the central nervous system (CNS) has been limited by inefficient gene delivery. AAV administration into the cerebrospinal fluid enables access to the CNS with relatively low doses and limited exposure to pre-existing anti-AAV antibodies. Previously the functional selection platform SIFTER (Selecting In vivo for Transduction and Expression of RNA) was applied to identify capsids with improved CNS transduction after cerebrospinal fluid (CSF) administration (see, US20200370137A1, which is incorporated by reference herein in its entirety). The engineered capsids STAC-102 and STAC-103 exhibited a 10- to 100-fold enrichment in both vector genome biodistribution and neuronal mRNA expression compared to AAV9 across key CNS regions. Fitness maturation of engineered capsids may further improve upon their key desirable properties. There remains a need to perform fitness maturation of capsid STAC-102 to identify second generation STAC-102 variants that mediate further improvement in CNS delivery in cynomolgus macaques, a key genetic and anatomical model of human CNS delivery.

### SUMMARY

STAC-102 is an engineered AAV capsid protein that has shown enhanced biodistribution across the central nervous system (CNS) compared to wild-type AAV9. Described herein are modifications to the STAC-102 engineered AAV capsid protein. In some embodiments, these modifications result in additional enhanced biodistribution across the CNS relative to the unmodified STAC-102 capsid protein. In some embodiments, the modifications comprise substituting specific amino acids within the STAC-102 engineered AAV capsid. In some embodiments, substituting specific amino acids within the STAC-102 capsid results in subsequences within the capsid known as STAC-102 variant sequences.

In some embodiments, an engineered AAV capsid protein is provided comprising at least 3, 4, 5, 6, 7, 8, 9, 10, 11, 12, or 13 contiguous amino acids of an amino acid sequence set forth in any one of SEQ ID NO: 1-6.

In an aspect, an engineered AAV capsid protein is provided comprising a diversified region as in STAC-102 variant sequences A-F. In an embodiment, variant A comprises SEQ ID NO: 1; variant B comprises SEQ ID NO: 2; variant C comprises SEQ ID NO: 3; variant D comprises SEQ ID NO: 4; variant E comprises SEQ ID NO: 5; or variant F comprises SEQ ID NO: 6.

In an aspect, an engineered AAV (e.g., STAC-102) sequence is provided comprising a variant diversified region SEQ ID NO: 1, wherein the diversified region comprises: at position 5, any one of the amino acids K, M, N, Y, P; and/or, at position 8, any one of the amino acids M, H, N, S, T, A, I, L, F, Y, P.

SEQ ID NO: 1

| | |
|---|---|
| Position | 1 2 3 4 5 6 7 8 9 10 11 12 13 |
| Amino Acid | R G N M K L T M Q E R Q A |

At position 5: Any of K, M, N, Y, P
At position 8: Any of M, H S, S, T, A, I, L, F, Y, P

In an aspect, an engineered AAV (e.g., STAC-102) sequence is provided comprising a variant diversified region SEQ ID NO: 2, wherein the diversified region comprises: at position 7, any one of the amino acids K, M, N, L; and/or, at position 8, any one of the amino acids S, Q, M.

SEQ ID NO: 2

| | |
|---|---|
| Position | 1 2 3 4 5 6 7 8 9 10 11 12 13 |
| Amino Acid | R G N M T L K S Q E R Q A |

At position 7: Any of K, M, N, L
At position 8: Any of S, Q, M

In an aspect, an engineered AAV (e.g., STAC-102) sequence is provided comprising a variant diversified region SEQ ID NO: 3, wherein the diversified region comprises: at position 4, any one of the amino acids K, R, F, Y, G; and/or, at position 9, any one of the amino acids D, K, N, S.

SEQ ID NO: 3

| | |
|---|---|
| Position | 1 2 3 4 5 6 7 8 9 10 11 12 13 |
| Amino Acid | R G N K T L T R D E R Q A |

At position 4: Any of K, R, F, Y, G
At position 9: Any of D, K, N, S

In an aspect, an engineered AAV (e.g., STAC-102) sequence is provided comprising a variant diversified region SEQ ID NO: 4, wherein the diversified region comprises: at position 11, any one of the amino acids K, S, L, Y, G, P; and/or, at position 12, any one of the amino acids T, D, E, V.

SEQ ID NO: 4

| | |
|---|---|
| Position | 1 2 3 4 5 6 7 8 9 10 11 12 13 |
| Amino Acid | R G N M T L T R Q E K T A |

At position 11: Any of K, S, L, Y, G, P
At position 12: Any of T, D, E, V

In an aspect, an engineered AAV (e.g., STAC-102) sequence is provided comprising a variant diversified region SEQ ID NO: 5, wherein the diversified region comprises: at position 4, any one of the amino acids T, M, D, E, Q, S, V, G; and/or, at position 7, any one of the amino acids P, H, R, A, V.

SEQ ID NO: 5

| | |
|---|---|
| Position | 1 2 3 4 5 6 7 8 9 10 11 12 13 |
| Amino Acid | R G N T T L P R Q E R Q A |

At position 4: Any of T, M, D, E, Q, S, V, G
At position 7: Any of P, H, R, A, V

In an aspect, an engineered AAV (e.g., STAC-102) sequence is provided comprising a variant diversified region SEQ ID NO: 6, wherein the diversified region comprises: at position 3, any one of the amino acids L, E, R, V; and/or, at position 4, any one of the amino acids T, H, E, A, P.

SEQ ID NO: 6

| | |
|---|---|
| Position | 1 2 3 4 5 6 7 8 9 10 11 12 13 |
| Amino Acid | R G L T T L T R Q E R Q A |

At position 3: Any of L, E, R, V
At position 4: Any of T, H, E, A, P

### BRIEF DESCRIPTION OF THE DRAWINGS

FIG. 1. Fitness maturation strategy. Each mutated position was changed to all possible amino acids except cysteine and the original amino acid in STAC-102. A total of ~9,000 capsid sequences were designed, and each was synthesized with three unique barcodes.

FIG. 2. Library design for functional capsid selection. Each capsid is linked to three unique barcodes using a bioinformatic look-up table. Hundreds to thousands of unique molecular identifiers (UMIs) are cloned per barcode to enable greater interpretation of distinct AAV transduction events. Capsid performance is evaluated based on barcoded mRNA expression from the neuron specific hSynapsin I promoter.

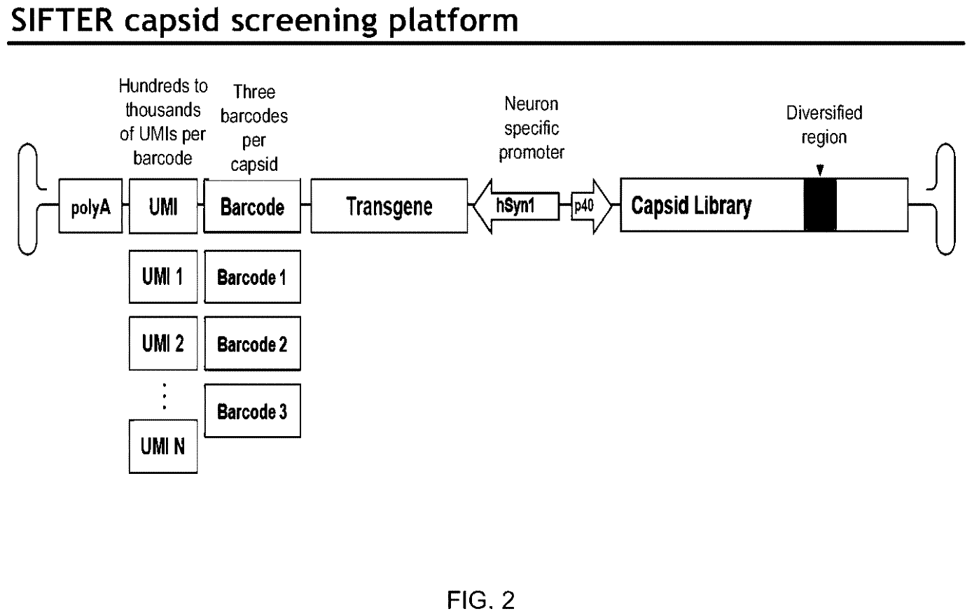

FIG. 3. Library evaluation of HEK293 manufacturing yield. High performing capsids have the following features in the bubble plots: (1) High log 2 fold enrichment (these data are normalized by input abundance (y-axis)); (2) Low coefficient of variation (x-axis); (3) High fraction of sequenced samples in which a capsid is found (large bubble size); (4) Robust unique molecular identifier recovery (green color). The parent capsid STAC-102 and notable second-generation capsids with improved performance in cynomolgus are annotated.

FIG. 4. In vitro transduction of mouse cortical neurons. Barcode expression in neurons was assessed 5 days post-transduction. Annotated second generation variants exhibit up to an 8-fold increase in mRNA expression relative to STAC-102.

FIG. 5. In vitro transduction of human iPSC-derived neurons. Barcode expression in neurons was assessed 9 days post-transduction. Annotated second generation variants exhibit up to a 25-fold increase in mRNA expression relative to STAC-102.

FIG. 6. Neuronal mRNA expression in whole brain slices. Two coronal slices from the forebrain and hindbrain were analyzed. Annotated second generation variants exhibit up to a 9-fold increase in mRNA expression relative to STAC-102.

FIG. 7. Neuronal mRNA expression in cortex brain punches. Brain punches were analyzed from 23 cortical regions and data were pooled. Annotated second generation variants exhibit up to a 10-fold increase in mRNA expression relative to STAC-102.

FIG. 8. Neuronal mRNA expression in the spinal cord. Certical, thoracic, and lumbar regions were analyzed. Annotated second generation variants exhibit up to an 8-fold increase in mRNA expression relative to STAC-102.

FIG. 9. Summary of second generation STAC-102 capsid performance. Fold change is calculated relative to STAC-102. Bubble size is proportional to fold change and UMI recovery is indicated by color. Six second generation variants were chosen based on their consistently high performance in cynomolgus macaque CNS tissues and manufacturing yield that is similar or better than the parent capsid STAC-102. Most of these variants likewise outperformed the STAC-102 parent in both mouse cortical neurons and human iPSC-derived neurons in vitro. Deep brain structures that are more difficult to transduce from the ICM route show a lower number of UMIs recovered.

FIG. 10. Heat map of capsids related to variant A (SEQ ID NO: 9). The log 2 fold change is for neuronal mRNA expression in cynomolgus macaque cortex tissue.

FIG. 11. Heat map of capsids related to variant B (SEQ ID NO: 10). The log 2 fold change is for neuronal mRNA expression in cynomolgus macaque cortex tissue.

FIG. 12. Heat map of capsids related to variant C (SEQ ID NO: 11). The log 2 fold change is for neuronal mRNA expression in cynomolgus macaque cortex tissue.

FIG. 13. Heat map of capsids related to variant D (SEQ ID NO: 12). The log 2 fold change is for neuronal mRNA expression in cynomolgus macaque cortex tissue.

FIG. 14. Heat map of capsids related to variant E (SEQ ID NO: 13). The log 2 fold change is for neuronal mRNA expression in cynomolgus macaque cortex tissue.

FIG. 15. Heat map of capsids related to variant F (SEQ ID NO: 14). The log 2 fold change is for neuronal mRNA expression in cynomolgus macaque cortex tissue.

### DETAILED DESCRIPTION

In an aspect, fitness maturation of the capsid STAC-102 resulted in identification of engineered STAC-102 variant sequences. In some embodiments, fitness maturation of the capsid STAC-102 resulted in identification of engineered STAC-102 variant sequences in Table 1. In some embodiments, the engineered STAC-102 variant sequences mediate enhanced delivery to the central nervous system (CNS).

In an aspect, an engineered AAV capsid protein is provided. In embodiments, the engineered AAV capsid protein comprises at least 3 contiguous amino acids from any of the diversified region sequences according to SEQ ID NO: 1, SEQ ID NO: 2, SEQ ID NO: 3, SEQ ID NO: 4, SEQ ID NO: 5, or SEQ ID NO: 6 described herein. In some embodiments, the engineered AAV capsid proteins comprise modifications of SEQ ID NO: 1-6, such as one or two substitutions to any amino acids within any of SEQ ID NO: 1-6.

In another aspect, an engineered STAC-102 AAV capsid protein is provided. In embodiments, the engineered STAC-102 capsid protein is at least 80%, 85%, 90%, 95%, or 99% identical to or comprises the sequence outside the diversified regions in any one of SEQ ID NO: 9 (variant sequence A), SEQ ID NO: 10 (variant sequence B), SEQ ID NO: 11 (variant sequence C), SEQ ID NO: 12 (variant sequence D), SEQ ID NO: 13 (variant sequence E), or SEQ ID NO: 14 (variant sequence F) described herein. In embodiments, the engineered STAC-102 capsid protein is at least 80%, 85%, 90%, 95%, or 99% identical to or comprises any one of SEQ ID NO: 9 (variant sequence A), SEQ ID NO: 10 (variant sequence B), SEQ ID NO: 11 (variant sequence C), SEQ ID NO: 12 (variant sequence D), SEQ ID NO: 13 (variant sequence E), or SEQ ID NO: 14 (variant sequence F) described herein. In some embodiments, the engineered STAC-102 capsid proteins comprise modifications of SEQ ID NO: 9-14, such as one or two substitutions to any amino acids within any of SEQ ID NO: 9-14.

### Engineered AAV Capsid Proteins

In an aspect, described herein are compositions and formulations comprising engineered AAV capsid proteins and methods of making and using the same. In some embodiments, the AAV capsid protein has enhanced tropism for a cell or tissue, e.g., for the delivery of genetic material to a specific cell or tissue, for example a CNS tissue or a CNS cell.

In some embodiments, the engineered AAV capsid proteins have advantages over wild-type AAV capsid proteins. In some embodiments, these advantages including (i) enhanced cell or tissue tropism as compared to the natural/wild-type AAV serotype, for example, enhanced cell or tissue tropism to the central nervous system (CNS) as compared to the natural/wild-type AAV serotype (ii) increased penetrance through the blood brain barrier following administration to a subject, (iii) wider distribution throughout the multiple brain regions, for example, the frontal cortex, sensory cortex, motor cortex, putamen, thalamus, cerebellar cortex, dentate nucleus, caudate, and/or hippocampus, (iv) elevated expression of genetic material in multiple brain regions. In some embodiments, the engineered AAV capsids enhance the delivery of genetic material to multiple regions of the brain including for example, the frontal cortex, sensory cortex, motor cortex, putamen, thalamus, cerebellar cortex, dentate nucleus, caudate, and/or hippocampus, (v) is delivery of genetic material of interest to a desired tissue, cell, or organelle.

In embodiments, the engineered AAV capsid proteins and genetic material described herein may be delivered to one or more (such as 1-10) target cells, tissues, organs, or organisms. In some embodiments, the engineered AAV capsid proteins have enhanced tropism for a specific target cell type, tissue or organ. As a non-limiting example, the engineered AAV capsid protein has enhanced tropism for cells and tissues of the central or peripheral nervous systems (CNS and PNS, respectively). In some embodiments, engineered AAV capsid proteins are produced recombinantly and are an adeno-associated virus (AAV) serotype such as AAV1, AAV2, AAV3B, AAV5, AAV6, AAV8, AAV9, AAV3, AAV4, AAV7, AAV11, AAVrh10, AAVrh39, or AAVrh74, or a combination thereof. In some embodiments, engineered AAV capsid proteins are produced recombinantly and are based on any one or more (such as 1-15) AAV serotypes known in the art. In some embodiments, a library of AAV variants comprises AAV variant capsid proteins derived from 2, 3, 4, 5, 6, 7, 8, 9, 10, 11, 12, 13, 14, 15, or more AAV serotypes. In some embodiments, the AAV variant capsid proteins derived from 2, 3, 4, 5, 6, 7, 8, 9, 10, 11, 12, 13, 14, 15, or more AAV serotypes are combined once individual serotype libraries are developed. In some embodiments, combinatorial libraries are generated by modifying nucleic acids encoding AAV capsid proteins from 2, 3, 4, 5, 6, 7, 8, 9, 10, 11, 12, 13, 14, 15, or more serotypes in the same pool.

Engineered AAV Capsid Proteins within a Cell

In some embodiments, engineered AAV capsid proteins are contained within a cell. In some embodiments, the cell is derived from the CNS. In some embodiments, the cell is derived from the PNS. In some embodiments, the cell is derived from the brain. In some embodiments, the cell is derived from the spinal cord. In some embodiments, the cell is derived from any of the frontal cortex, the sensory cortex, the motor cortex, the cerebellar cortex, the cerebral cortex, the brain stem, the hippocampus, or the thalamus, amongst others.

### Engineered AAV Capsid Proteins Delivered to a Target Cell

The engineered AAV capsid proteins may be delivered to one or more target cells, tissues, organs, or organisms. In some embodiments, the engineered AAV capsid proteins demonstrate enhanced tropism for a target cell type, tissue or organ. As a non-limiting example, the engineered AAV capsid proteins may have enhanced tropism for cells and tissues of the central or peripheral nervous systems, or cells and tissues of a muscle. The engineered AAV capsid proteins may, in addition, or alternatively, have decreased tropism for an undesired target cell-type, tissue or organ. As a non-limiting example, the engineered AAV capsid proteins may have enhanced tropism for B cells, hematopoietic cells, leukocytes, platelets, macrophages, megakaryocytes, monocytes and/or T cells.

### Fitness Maturation of Modified STAC-102 Parent Capsids

In some embodiments, the engineered AAV capsid protein comprises a modified STAC-102 parent capsid. In embodiments, the engineered STAC-102 capsid protein is at least 80%, 85%, 90%, 95%, or 99% identical to or comprises the sequence of SEQ ID NO: 8. In some embodiments, the engineered AAV capsid protein comprises a modified STAC-102 parent capsid comprising one or two amino acid modifications to SEQ ID NO: 8.

(SEQ ID NO: 8)
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNMTLTRQERQAATA
DVNTQGVLPGMVWQDRDVYLQGPIWAKIPHTDGHFHPSPLMGGFGLKHPP
PQILIKNTPVPANPSTTFSAAKFASFITQYSTGQVSVEIEWELQKENSKR
WNPEIQYTSNYNKSVNVDFTVDINGVYSEPRPIGTRYLTRNL

In some embodiments, the one or two modifications to SEQ ID NO: 8 comprises one or two substitutions to SEQ ID NO: 8. In some embodiments, the one or two substitutions of SEQ ID NO: 8 occurs at one or two of any of the following the amino acids 585, 586, 587, 588, 589, 590, 591, 592, 593, 594, 595, 596, and 597 (amino acids shown in bold in SEQ ID NO: 8). In some embodiments, amino acids 585-597 of the STAC-102 capsid comprise the diversified region of the STAC-102 parent capsid. In some embodiments, the diversified region of the STAC-102 parent capsid comprises the sequence: RGNMTLTRQERQA (SEQ ID NO: 7).

### Modifications to Amino Acids in SEQ ID NO: 8 (STAC-102 Parent Capsid)

In some embodiments, the STAC-102 parent capsid is modified, i.e., comprises a substitution, insertion, deletion or combinations thereof. In some embodiments, the STAC-102 parent capsid is modified at amino acid position 585. In some embodiments, the modification at position 585 comprises a substitution. In some embodiments, the STAC-102 parent capsid is modified at amino acid position 586. In some embodiments, the modification at position 586 comprises a substitution. In some embodiments, the STAC-102 parent capsid is modified at amino acid position 587. In some embodiments, the modification at position 587 comprises a substitution. In some embodiments, the STAC-102 parent capsid is modified at amino acid position 588. In some embodiments, the modification at position 588 comprises a substitution. In some embodiments, the diversified region of the STAC-102 parent capsid is modified at amino acid position 589. In some embodiments, the modification at position 589 comprises a substitution. In some embodiments, the diversified region of the STAC-102 parent capsid is modified at amino acid position 590. In some embodiments, the modification at position 590 comprises a substitution. In some embodiments, the STAC-102 parent capsid is modified at amino acid position 591. In some embodiments, the modification at position 591 comprises a substitution. In some embodiments, the STAC-102 parent capsid is modified at amino acid position 592. In some embodiments, the modification at position 592 comprises a substitution. In some embodiments, the STAC-102 parent capsid is modified at amino acid position 593. In some embodiments, the modification at position 593 comprises a substitution. In some embodiments, the STAC-102 parent capsid is modified at amino acid position 594. In some embodiments, the modification at position 594 comprises a substitution. In some embodiments, the STAC-102 parent capsid is modified at amino acid position 595. In some embodiments, the modification at position 595 comprises a substitution. In some embodiments, the diversified region of the STAC-102 parent capsid is modified at amino acid position 596. In some embodiments, the modification at position 596 comprises a substitution. In some embodiments, the diversified region of the STAC-102 parent capsid is modified at amino acid position 596. In some embodiments, the modification at position 596 comprises a substitution. In some embodiments, the STAC-102 parent capsid is modified at amino acid position 597. In some embodiments, the modification at position 597 comprises a substitution. In some embodiments, the STAC-102 parent capsid is modified at amino acid position 585, 586, 587, 588, 589, 590, 591, 592, 593, 594, 595, 596, and/or 597. In some embodiments, the modification at position 585, 586, 587, 588, 589, 590, 591, 592, 593, 594, 595, 596, and/or 597 comprises a substitution, insertion, deletion or combinations thereof.

Disclosed are combinations of positions that can be modified in the diversified region of the STAC-102 parent capsid. Note, that the amino acids referenced here are from the perspective of the STAC-102 parent capsid, not from the perspective of the engineered STAC-102 variant sequences. In some embodiments, the STAC-102 parent capsid is modified at amino acid positions 589 and 592. In some embodiments, the modifications at positions 589 and 592, comprise one substitution at each of positions 589 and 592. In some embodiments, the STAC-102 parent capsid is modified at amino acid positions 591 and 592. In some embodiments, the modifications at positions 591 and 592 comprise one substitution at each of positions 591 and 592. In some embodiments, the STAC-102 parent capsid is modified at amino acid positions 588 and 593. In some embodiments, the modifications at positions 588 and 593, comprise one substitution at each of positions 588 and 593. In some embodiments, the STAC-102 parent capsid is modified at amino acid positions 595 and 596. In some embodiments, the modifications at positions 595 and 596, comprise one substitution at each of positions 595 and 596. In some embodiments, the STAC-102 parent capsid is modified at amino acid positions 588 and 591. In some embodiments, the modifications at positions 588 and 591 comprise one substitution at each of positions 588 and 591. In some embodiments, the STAC-102 parent capsid is modified at amino acid positions 587 and 588. In some embodiments, the modifications at positions 587 and 588 comprise one substitution at each of positions 587 and 588.

In some embodiments, the STAC-102 parent capsid is modified at amino acid positions 585 and 586. In some embodiments, the modifications at positions 585 and 586, comprise one substitution at each of positions 585 and 586. In some embodiments, the STAC-102 parent capsid is modified at amino acid positions 585 and 587. In some embodiments, the modifications at positions 585 and 587, comprise one substitution at each of positions 585 and 587. In some embodiments, the STAC-102 parent capsid is modified at amino acid positions 585 and 588. In some embodiments, the modifications at positions 585 and 588, comprise one substitution at each of positions 585 and 588. In some embodiments, the STAC-102 parent capsid is modified at amino acid positions 585 and 589. In some embodiments, the modifications at positions 585 and 589, comprise one substitution at each of positions 585 and 589, In some embodiments, the STAC-102 parent capsid is modified at amino acid positions 585 and 590. In some embodiments, the modifications at positions 585 and 590, comprise one substitution at each of positions 585 and 590. In some embodiments, the STAC-102 parent capsid is modified at amino acid positions 585 and 591. In some embodiments, the modifications at positions 585 and 591, comprise one substitution at each of positions 585 and 591. In some embodiments, the STAC-102 parent capsid is modified at amino acid positions 585 and 592. In some embodiments, the modifications at positions 585 and 592, comprise one substitution at each of positions 585 and 592. In some embodiments, the STAC-102 parent capsid is modified at amino acid positions 585 and 593. In some embodiments, the modifications at positions 585 and 593, comprise one substitution at each of positions 585 and 593. In some embodiments, the STAC-102 parent capsid is modified at amino acid positions 585 and 594. In some embodiments, the modifications at positions 585 and 594, comprise one substitution at each of positions 585 and 594. In some embodiments, the STAC-102 parent capsid is modified at amino acid positions 585 and 595. In some embodiments, the modifications at positions 585 and 595, comprise one substitution at each of positions 585 and 595. In some embodiments, the STAC-102 parent capsid is modified at amino acid positions 585 and 596. In some embodiments, the modifications at positions 585 and 596, comprise one substitution at each of positions 585 and 596. In some embodiments, the STAC-102 parent capsid is modified at amino acid positions 585 and 597. In some embodiments, the modifications at positions 585 and 597, comprise one substitution at each of positions 585 and 597. In some embodiments, the STAC-102 parent capsid is modified at amino acid positions 586 and 587. In some embodiments, the modifications at positions 586 and 587, comprise one substitution at each of positions 586 and 587. In some embodiments, the STAC-102 parent capsid is modified at amino acid positions 586 and 588. In some embodiments, the modifications at positions 586 and 588, comprise one substitution at each of positions 586 and 588. In some embodiments, the STAC-102 parent capsid is modified at amino acid positions 586 and 589. In some embodiments, the modifications at positions 586 and 589, comprise one substitution at each of positions 586 and 589. In some embodiments, the STAC-102 parent capsid is modified at amino acid positions 586 and 590. In some embodiments, the modifications at positions 586 and 590, comprise one substitution at each of positions 586 and 590. In some embodiments, the STAC-102 parent capsid is modified at amino acid positions 586 and 591. In some embodiments, the modifications at positions 586 and 591, comprise one substitution at each of positions 586 and 591. In some embodiments, the STAC-102 parent capsid is modified at amino acid positions 586 and 592. In some embodiments, the modifications at positions 586 and 592, comprise one substitution at each of positions 586 and 592. In some embodiments, the STAC-102 parent capsid is modified at amino acid positions 586 and 593. In some embodiments, the modifications at positions 586 and 593, comprise one substitution at each of positions 586 and 593. In some embodiments, the STAC-102 parent capsid is modified at amino acid positions 586 and 594. In some embodiments, the modifications at positions 586 and 594, comprise one substitution at each of positions 586 and 594. In some embodiments, the STAC-102 parent capsid is modified at amino acid positions 586 and 595. In some embodiments, the modifications at positions 586 and 595, comprise one substitution at each of positions 586 and 595. In some embodiments, the STAC-102 parent capsid is modified at amino acid positions 586 and 596. In some embodiments, the modifications at positions 586 and 596, comprise one substitution at each of positions 586 and 596. In some embodiments, the STAC-102 parent capsid is modified at amino acid positions 586 and 597. In some embodiments, the modifications at positions 586 and 597, comprise one substitution at each of positions 586 and 597. In some embodiments, the STAC-102 parent capsid is modified at amino acid positions 587 and 588. In some embodiments, the modifications at positions 587 and 588, comprise one substitution at each of positions 587 and 588. In some embodiments, the STAC-102 parent capsid is modified at amino acid positions 587 and 589. In some embodiments, the modifications at positions 587 and 589, comprise one substitution at each of positions 587 and 589. In some embodiments, the STAC-102 parent capsid is modified at amino acid positions 587 and 590. In some embodiments, the modifications at positions 587 and 590, comprise one substitution at each of positions 587 and 590. In some embodiments, the STAC-102 parent capsid is modified at amino acid positions 587 and 591. In some embodiments, the modifications at positions 587 and 591, comprise one substitution at each of positions 587 and 591. In some embodiments, the STAC-102 parent capsid is modified at amino acid positions 587 and 592. In some embodiments, the modifications at positions 587 and 592, comprise one substitution at each of positions 587 and 592. In some embodiments, the STAC-102 parent capsid is modified at amino acid positions 587 and 593. In some embodiments, the modifications at positions 587 and 593, comprise one substitution at each of positions 587 and 593. In some embodiments, the STAC-102 parent capsid is modified at amino acid positions 587 and 594. In some embodiments, the modifications at positions 587 and 594, comprise one substitution at each of positions 587 and 594. In some embodiments, the STAC-102 parent capsid is modified at amino acid positions 587 and 595. In some embodiments, the modifications at positions 587 and 595, comprise one substitution at each of positions 587 and 595. In some embodiments, the STAC-102 parent capsid is modified at amino acid positions 587 and 586. In some embodiments, the modifications at positions 587 and 596, comprise one substitution at each of positions 587 and 596. In some embodiments, the STAC-102 parent capsid is modified at amino acid positions 587 and 597. In some embodiments, the modifications at positions 587 and 597, comprise one substitution at each of positions 587 and 597. In some embodiments, the STAC-102 parent capsid is modified at amino acid positions 588 and 589. In some embodiments, the modifications at positions 588 and 589, comprise one substitution at each of positions 588 and 589. In some embodiments, the STAC-102 parent capsid is modified at amino acid positions 588 and 590. In some embodiments, the modifications at positions 588 and 590, comprise one substitution at each of positions 588 and 590. In some embodiments, the STAC-102 parent capsid is modified at amino acid positions 588 and 591. In some embodiments, the modifications at positions 588 and 591, comprise one substitution at each of positions 588 and 591. In some embodiments, the STAC-102 parent capsid is modified at amino acid positions 588 and 592. In some embodiments, the modifications at positions 588 and 592, comprise one substitution at each of positions 588 and 592. In some embodiments, the STAC-102 parent capsid is modified at amino acid positions 588 and 593. In some embodiments, the modifications at positions 588 and 593, comprise one substitution at each of positions 588 and 593. In some embodiments, the STAC-102 parent capsid is modified at amino acid positions 588 and 594. In some embodiments, the modifications at positions 588 and 594, comprise one substitution at each of positions 588 and 594. In some embodiments, the STAC-102 parent capsid is modified at amino acid positions 588 and 595. In some embodiments, the modifications at positions 588 and 595, comprise one substitution at each of positions 588 and 595. In some embodiments, the STAC-102 parent capsid is modified at amino acid positions 588 and 596. In some embodiments, the modifications at positions 588 and 596, comprise one substitution at each of positions 588 and 596. In some embodiments, the STAC-102 parent capsid is modified at amino acid positions 588 and 597. In some embodiments, the modifications at positions 588 and 597, comprise one substitution at each of positions 588 and 597. In some embodiments, the STAC-102 parent capsid is modified at amino acid positions 589 and 590. In some embodiments, the modifications at positions 589 and 590, comprise one substitution at each of positions 589 and 590. In some embodiments, the STAC-102 parent capsid is modified at amino acid positions 589 and 591. In some embodiments, the modifications at positions 589 and 591, comprise one substitution at each of positions 589 and 591. In some embodiments, the STAC-102 parent capsid is modified at amino acid positions 589 and 592. In some embodiments, the modifications at positions 589 and 592, comprise one substitution at each of positions 589 and 592. In some embodiments, the STAC-102 parent capsid is modified at amino acid positions 589 and 593. In some embodiments, the modifications at positions 589 and 593, comprise one substitution at each of positions 589 and 593. In some embodiments, the STAC-102 parent capsid is modified at amino acid positions 589 and 594. In some embodiments, the modifications at positions 589 and 594, comprise one substitution at each of positions 589 and 594. In some embodiments, the STAC-102 parent capsid is modified at amino acid positions 589 and 595. In some embodiments, the modifications at positions 589 and 595, comprise one substitution at each of positions 589 and 595. In some embodiments, the STAC-102 parent capsid is modified at amino acid positions 589 and 596. In some embodiments, the modifications at positions 589 and 596, comprise one substitution at each of positions 589 and 596. In some embodiments, the STAC-102 parent capsid is modified at amino acid positions 589 and 597. In some embodiments, the modifications at positions 589 and 597, comprise one substitution at each of positions 589 and 597. In some embodiments, the STAC-102 parent capsid is modified at amino acid positions 590 and 591. In some embodiments, the modifications at positions 590 and 591, comprise one substitution at each of positions 590 and 591. In some embodiments, the STAC-102 parent capsid is modified at amino acid positions 590 and 592. In some embodiments, the modifications at positions 590 and 592, comprise one substitution at each of positions 590 and 592. In some embodiments, the STAC-102 parent capsid is modified at amino acid positions 590 and 593. In some embodiments, the modifications at positions 590 and 593, comprise one substitution at each of positions 590 and 593. In some embodiments, the STAC-102 parent capsid is modified at amino acid positions 590 and 594. In some embodiments, the modifications at positions 590 and 594, comprise one substitution at each of positions 590 and 594. In some embodiments, the STAC-102 parent capsid is modified at amino acid positions 590 and 595. In some embodiments, the modifications at positions 590 and 595, comprise one substitution at each of positions 590 and 595. In some embodiments, the STAC-102 parent capsid is modified at amino acid positions 590 and 596. In some embodiments, the modifications at positions 590 and 596, comprise one substitution at each of positions 590 and 596. In some embodiments, the STAC-102 parent capsid is modified at amino acid positions 590 and 597. In some embodiments, the modifications at positions 590 and 597, comprise one substitution at each of positions 590 and 597. In some embodiments, the STAC-102 parent capsid is modified at amino acid positions 591 and 592. In some embodiments, the modifications at positions 591 and 592, comprise one substitution at each of positions 591 and 592. In some embodiments, the STAC-102 parent capsid is modified at amino acid positions 591 and 593. In some embodiments, the modifications at positions 591 and 593, comprise one substitution at each of positions 591 and 593. In some embodiments, the STAC-102 parent capsid is modified at amino acid positions 591 and 594. In some embodiments, the modifications at positions 591 and 594, comprise one substitution at each of positions 591 and 594. In some embodiments, the STAC-102 parent capsid is modified at amino acid positions 591 and 595. In some embodiments, the modifications at positions 591 and 595, comprise one substitution at each of positions 591 and 595. In some embodiments, the STAC-102 parent capsid is modified at amino acid positions 591 and 596. In some embodiments, the modifications at positions 591 and 596, comprise one substitution at each of positions 591 and 596. In some embodiments, the STAC-102 parent capsid is modified at amino acid positions 591 and 597. In some embodiments, the modifications at positions 591 and 597, comprise one substitution at each of positions 591 and 597. In some embodiments, the STAC-102 parent capsid is modified at amino acid positions 592 and 593. In some embodiments, the modifications at positions 592 and 593, comprise one substitution at each of positions 592 and 593. In some embodiments, the STAC-102 parent capsid is modified at amino acid positions 592 and 594. In some embodiments, the modifications at positions 592 and 594, comprise one substitution at each of positions 592 and 594. In some embodiments, the STAC-102 parent capsid is modified at amino acid positions 592 and 595. In some embodiments, the modifications at positions 592 and 595, comprise one substitution at each of positions 592 and 595. In some embodiments, the STAC-102 parent capsid is modified at amino acid positions 592 and 596. In some embodiments, the modifications at positions 592 and 596, comprise one substitution at each of positions 592 and 596. In some embodiments, the STAC-102 parent capsid is modified at amino acid positions 592 and 597. In some embodiments, the modifications at positions 592 and 597, comprise one substitution at each of positions 592 and 597. In some embodiments, the STAC-102 parent capsid is modified at amino acid positions 593 and 594. In some embodiments, the modifications at positions 593 and 594, comprise one substitution at each of positions 593 and 594. In some embodiments, the STAC-102 parent capsid is modified at amino acid positions 593 and 595. In some embodiments, the modifications at positions 593 and 595, comprise one substitution at each of positions 593 and 595. In some embodiments, the STAC-102 parent capsid is modified at amino acid positions 593 and 596. In some embodiments, the modifications at positions 593 and 596, comprise one substitution at each of positions 593 and 596. In some embodiments, the STAC-102 parent capsid is modified at amino acid positions 593 and 597. In some embodiments, the modifications at positions 593 and 597, comprise one substitution at each of positions 593 and 597. In some embodiments, the STAC-102 parent capsid is modified at amino acid positions 594 and 595. In some embodiments, the modifications at positions 594 and 595, comprise one substitution at each of positions 594 and 595. In some embodiments, the STAC-102 parent capsid is modified at amino acid positions 594 and 596. In some embodiments, the modifications at positions 594 and 596, comprise one substitution at each of positions 594 and 596. In some embodiments, the STAC-102 parent capsid is modified at amino acid positions 594 and 597. In some embodiments, the modifications at positions 594 and 597, comprise one substitution at each of positions 594 and 597. In some embodiments, the STAC-102 parent capsid is modified at amino acid positions 595 and 596. In some embodiments, the modifications at positions 595 and 596, comprise one substitution at each of positions 595 and 596. In some embodiments, the STAC-102 parent capsid is modified at amino acid positions 595 and 597. In some embodiments, the modifications at positions 595 and 597, comprise one substitution at each of positions 595 and 597. In some embodiments, the STAC-102 parent capsid is modified at amino acid positions 596 and 597. In some embodiments, the modifications at positions 596 and 597, comprise one substitution at each of positions 596 and 597. In some embodiments, the one or modifications to SEQ ID NO: 8 results in any one of the sequences set forth in SEQ ID NO: 9-14.

### Engineered AAV Capsid Protein Library Generation

In one aspect, disclosed herein is the development of libraries encoding engineered AAV capsid proteins with a desired characteristic compared to a natural/wild-type AAV serotype. In one aspect, disclosed herein is the development of libraries encoding engineered AAV capsid proteins with a desired characteristic compared to the parent capsid STAC-102. Thus, described herein are libraries of AAV capsid proteins with a desired characteristic compared to the parent capsid STAC-102. In some embodiments, the desired characteristic is enhanced cell or tissue tropism as compared to the parent capsid STAC-102, for example, enhanced cell or tissue tropism to the central nervous system (CNS) as compared to the parent capsid STAC-102. In some embodiments, the desired characteristic is increased penetrance through the blood brain barrier following administration to a subject. In some embodiments, the desired characteristic is wider distribution throughout the multiple brain regions, e.g., frontal cortex, sensory cortex, motor cortex, putamen, thalamus, cerebellar cortex, dentate nucleus, caudate, and/or hippocampus. In some embodiments, the desired characteristic is elevated genetic material expression in multiple brain regions. In some embodiments, the desired characteristic is delivery of genetic material of interest to a desired tissue, cell, or organelle.

In some embodiments, each member of a library comprises one or more of a) a nucleic acid sequence encoding an AAV capsid protein comprising an engineered STAC-102 variant sequence; b) a nucleic acid sequence encoding barcode; c) nucleic acid sequence(s) encoding a promoter(s); d) a nucleic acid sequence encoding a unique molecular identifier (UMI); and combinations thereof. In some embodiments, each member of the library also includes genetic material to be delivered to a cell or tissue of interest. In some embodiments, each member of the library also includes a polyA sequence.

In some embodiments, each engineered AAV capsid protein was synthesized as an oligo pool. In some embodiments, each member of a library comprises one or more (such as 1-10) of a nucleic acid sequence encoding an AAV capsid protein comprising a) a nucleic acid sequence encoding one or more (such as 1-10) barcodes: b) one or more (such as 1-10) nucleic acid sequences encoding one or more (such as 1-10) promoters; c) a nucleic acid sequence encoding a unique molecular identifier (UMI); or combinations thereof. In some embodiments, each member of the library also includes genetic material to be delivered to a cell or tissue of interest. In some embodiments, each member of the library also includes a polyA sequence. In some embodiments, each of the one or more (such as 1-10) barcodes is linked to the identity of a single engineered AAV capsid protein. In some embodiments, each of the barcodes is linked to the identity of a single engineered AAV capsid protein. In some embodiments, each of the barcodes is linked to a UMI.

In some embodiments, a nucleic acid comprising a barcode is added to the genome of each AAV capsid proteins in a library. In some embodiments, the barcode is bioinformatically linked to the STAC-102 variant sequence. In some embodiments, the DNA sequences encoding the STAC-102 variant sequence are synthesized to further comprise a random or specified barcode. The barcode may comprise 5, 6, 7, 8, 9, 10, 11, 12, 13, 14, 15, 16, 17, 18 or more nucleotides. In some embodiments, each targeting STAC-102 variant sequence is linked to at least 2 distinct barcodes. In some embodiments, each barcode is linked to one or more (such as 1-10) UMI.

In some embodiments, each member of the library comprises a nucleic acid comprising more than one barcode sequences (such as 1-10). In some embodiments, each member of the library comprises two or more nucleic acids (such as 1-10) each comprising a barcode sequence. In some embodiments, each member of the library comprises a first nucleic acid comprising a first barcode and a second nucleic acid comprising a second barcode. In some embodiments, the first nucleic acid comprising the first barcode and the second nucleic acid comprising the second barcode are different. In some embodiments, each of the first nucleic acid comprising the first barcode and the second nucleic acid comprising the second barcode is independently operatively linked to a promoter. In some embodiments, each capsid is linked to one to three unique barcodes using a bioinformatic look-up table. In some embodiments, a capsid is linked to three unique barcodes and 100-1,000 or 100-2,000 or 100-5,000 or 100-10,000 UMIs are cloned per barcode to enable greater interpretation of distinct AAV transduction events. In some embodiments, capsid performance is evaluated based on barcoded mRNA expression from the neuron specific promoter. In some embodiments, capsid performance is evaluated based on barcoded mRNA expression from the neuron specific hSynapsin I promoter.

In some embodiments, libraries were created containing engineered AAV capsid proteins that comprise at least one mutation relative to the parent capsid STAC-102. In some embodiments, the engineered AAV capsid proteins contained two mutations relative to the parent capsid STAC-102. In some embodiments, the engineered AAV capsid proteins contained 1-10 mutations relative to the parent capsid STAC-102. In some embodiments, each mutated position was changed to all possible amino acids except cysteine and the original amino acid in STAC-102.

In some embodiments, the libraries are packaged in HEK293 cells where the helper functions (e.g. E2A, E4, VA, E1A and E1B) are supplied in trans. In some embodiments, the AA V rep function comprises rep78, rep 68, rep 52, and rep40 genes. In some embodiments, the rep genes are supplied in trans. In some embodiments, the start codon of the rep78 and/or the rep68 gene is altered from ACG to ATG to increase replication of the capsid library construct containing inverted terminal repeats (ITRs), thereby improving AAV library manufacturing yield. In some embodiments, the cap genes are supplied as genetic material to the manufactured AAVs. In some embodiments, the capsid gene is controlled by the p40 promoter such that it is only expressed during manufacturing in HEK293 cells in the presence of helper virus functions.

### Performance of Engineered AAV Capsid Proteins

In some embodiments, high performing capsids have the following features in the bubble plots: (1) High log 2 fold enrichment (these data are normalized by input abundance (y-axis)); (2) Low coefficient of variation (x-axis); (3) High fraction of sequenced samples in which a capsid is found (large bubble size); (4) Robust unique molecular identifier recovery (green color); (5) and combinations thereof.

In some embodiments, barcode expression in neurons was assessed 1-5, or 1-10, or 5-10, or 5 days post-transduction. In some embodiments, second generation variants (i.e. capsids having a mutation or 1-2 or 1-10 mutations relative to the parent capsid STAC-102) exhibit up to a 5 to 25-fold increase in mRNA expression relative to the parent STAC-102. In some embodiments, coronal slices from the forebrain and/or hindbrain were analyzed for mRNA expression of modified STAC-102 parent capsid relative to the parent capsid STAC-102. In some embodiments, second generation variants (i.e. capsids having a mutation or 1-2 or 1-10 mutations relative to the parent capsid STAC-102) exhibit a 2-15 fold increase in mRNA expression relative to parent capsid STAC-102. In some embodiments, brain punches from cortical regions were pooled and mRNA expression for second generation variants relative to the parent STAC-102 was assessed. Annotated second generation variants exhibit up to a 5-20 fold increase in mRNA expression relative to parent STAC-102. In some embodiments, mRNA expression for second generation variants relative to the parent STAC-102 in certical, thoracic, and lumbar regions were analyzed. Second generation variants exhibit up to an 5-15 fold increase in mRNA expression relative to STAC-102.

### Adeno-Associated Virus (AAV)

AAV are capable of infecting a wide range of cells including quiescent cells and dividing cells. In some embodiments, In some embodiments, AAV can be modified to so that it contains the components necessary for the assembly of a functional recombinant virus or viral particle. In some embodiments, AAV is engineered to target a specific tissue and/or cell. In some embodiments, the AAV is engineered to deliver a specific genetic material to a tissue and/or cell.

### Modified AAV Serotypes

In some embodiments, the AAV is based on any natural or recombinant AAV serotype. Different AAV serotypes have different characteristics such as different packaging, tropism, and transduction profiles. In some embodiments, the engineered AAV capsid proteins are based on a wild-type AAV serotype. In some embodiments, the AAV serotype comprises AAV1, AAV2, AAV3B, AAV5, AAV6, AAV8, or AAV9. In some embodiments, the AAV serotype comprises less well-characterized AAV serotypes such as AAV3, AAV4, AAV7, AAV11, AAVrh10, AAVrh39, or AAVrh74. In some embodiments, the engineered AAV capsid protein derives from a multiple wild-type AAV serotypes, for example, 2, 3, 4, 5, 6, 7, 8, 9, 10, 11, 12, 13, 14, 15, or more AAV serotypes. In some embodiments, the AAV variant capsid proteins derived from 2, 3, 4, 5, 6, 7, 8, 9, 10, 11, 12, 13, 14, 15, or more AAV serotypes are combined once individual serotype libraries are developed. In some embodiments, combinatorial libraries are generated by modifying nucleic acids encoding AAV capsid proteins from 2, 3, 4, 5, 6, 7, 8, 9, 10, 11, 12, 13, 14, 15, or more serotypes in the same pool.

In some embodiments, different AAV serotypes are different in their ability to direct or modulate an AAV particle to a particular cell or tissue. In some embodiments, the AAV serotype can be modified to increase the tropism of the AAV particle to cells or tissues of the central nervous system (CNS). In some embodiments, the AAV serotype can be modified to increase tropism of the AAV particle to cells or tissues of the peripheral nervous system (PNS).

In some embodiments, the modified AAV serotype has a desired characteristic compared to a natural/wild-type AAV serotype. In some embodiments, the modified AAV serotype allows for increased penetration of penetration of the blood brain barrier following administration to a subject. In some embodiments, the modified AAV serotype causes increased biodistribution to a brain region. In some embodiments, the brain region comprises the frontal cortex, the sensory cortex, the motor cortex, the cerebellar cortex, the hippocampus, the thalamus, or the putamen. In some embodiments, the brain comprises any brain region known in the art. In some embodiments, the modified AAV serotype causes increased biodistribution to more than one brain regions, for example, 2 brain regions, 3 brain regions, 4 brain regions, 5 brain regions, 6 brain regions, 7 brain regions, 8 brain regions, 9 brain regions, or 10 brain regions. In some embodiments, the modified AAV serotype causes increased biodistribution to 1-10 brain regions. In some embodiments, the modified AAV serotype are useful in elevating genetic material expression in multiple brain regions. In some embodiments, the modified AAV serotype are used to deliver genetic material of interest to a desired tissue, cell, or organelle.

In some embodiments, the modified AAV serotype causes increased biodistribution to regions of the spinal cord. In some embodiments, the region of the spinal cord comprises any of the thoracic spinal cord region, the lumbar spinal cord region, and/or the cervical spinal cord region. In some embodiments, the region of the spinal cord includes any region of the spinal cord known in the art. In some embodiments, the modified AAV serotype cases increased biodistribution to more than one region of the spinal cord, for example, 2 spinal cord regions, 3 spinal cord regions, 4 spinal cord regions, 5 spinal cord regions, 6 spinal cord regions, 7 spinal cord regions, 8 spinal cord regions, 9 spinal cord regions, or 10 spinal cord regions. In some embodiments, the modified AAV serotype cases increased biodistribution to 1-10 spinal cord regions.

In some embodiments, the modified AAV serotype is modified with a peptide that comprises at least 85%, 86%, 87%, 88%, 89%, 90%, 91%, 92%, 93%, 94%, 95%, 96%, 97%, 98%, or 99% sequence identity of any peptide (i.e., variant sequence) set forth in any of SEQ ID NO: 1-6. In some embodiments, the modified AAV serotype is modified with a peptide (i.e., variant sequence) set forth in any of SEQ ID NO: 1-6. In some embodiments, the modified AAV serotype is modified with any of the peptides described herein.

### Structure of AAV

In some embodiments, the genome of the AAV comprises a single-strand DNA (ssDNA) molecule that is approximately between about 4.5 kb and about 5.0 kb in length, for example about 4.5 kb, about 4.6 kb, about 4.7 kb, about 4.8 kb, about 4.9 kb, or about 5.0 kb in length. In some embodiment, the AAV genome contains inverted terminal repeats (ITRs) that flank the 5′ and 3′ ends of the AAV molecule. In some embodiments, the ITRs contain origins of replication for the viral genome. In some embodiments, the length of the ITRs is about 145 bp in length, for example, between about 130 bp in length and 160 bp in length.

In some embodiments, the AAV genome comprises at least three genes. In some embodiments, the at least three genes comprise rep, cap, and X. In some embodiments, the AAV genome nucleotide includes nucleotide sequences that encode four non-structural Rep proteins (Rep 78, Rep68, Rep52, Rep40, encoded by Rep genes). In some embodiments, the AAV viral genome includes nucleotide sequences that encode the three capsid, or structural, proteins (i.e., VP1, VP2, VP3, encoded by capsid genes or cap genes). In some embodiments, the rep proteins are used for replication and packaging. In some embodiments, the capsid proteins are assembled to create the protein shell of the AAV.

### AAV Particles

In some embodiments, the engineered AAV capsid proteins are packaged into AAV particles. In some embodiments, the AAV particles that have enhanced tropism for a target tissue (e.g., CNS and PNS) are provided. In some embodiments, the AAV particles include engineered STAC-102 variant sequences that alter tropism to a particular cell-type, tissue, organ or organism, in vivo, ex vivo or in vitro. In some embodiments, the AAV particles are capable of penetrating the blood brain barrier.

### Delivery of AAV Particles

The AAV particles may be delivered to one or more target cells, tissues, organs, or organisms. In some embodiments, the AAV particles demonstrate enhanced tropism for a target cell type, tissue or organ. As a non-limiting example, the AAV particle may have enhanced tropism for cells and tissues of the central or peripheral nervous systems (CNS and PNS, respectively), or cells and tissues of a muscle. The AAV particles may, in addition, or alternatively, have decreased tropism for an undesired target cell-type, tissue or organ.

In some embodiments, the AAV particles are used to deliver a viral genome to a tissue or cells such as CNS or PNS cell or tissue.

The delivered viral genome may include genetic material of interest, such as, for example, genetic material that encodes an antibody or an enzyme, or regulatory RNA, amongst others. In some embodiments, the viral genome includes at least one ITR sequence. In some embodiments, the viral genome includes 2 ITR sequences. In some embodiments, the ITR sequences flank the genetic material of interest. In some embodiments, the ITR sequences are complementary to each other. In some embodiments, the ITR regions are derived from the same serotype as the capsid protein. ITR regions may be between 100 and 150 nucleotides in length.

In some embodiments, the AVV particles can be used to infect a wide range of cells (including quiescent and dividing cells) without integration into the host genome and without replicating. In some embodiments, the genome of the virus contains the components required for the assembly of a functional recombinant virus, or viral particle, which is loaded with or engineered to target a particular tissue and express or deliver genetic material of interest to the particular tissue.

### AAV Diversified Region Sequences

In embodiments, sequences of AAV diversified regions disclosed herein. In some embodiments, the sequences can function as a general CNS-targeting molecule.

In some embodiments, the CNS-targeting molecule is fused or conjugated to a small molecule, an antibody, scFV, ASO (antisense oligonucleotide), siRNA, lipid, polymer or recombinant protein. In some embodiments, any of SEQ ID NO: 1-6 is fused or conjugated to a small molecule, an antibody, scFV, ASO (antisense oligonucleotide), siRNA, lipid, polymer or recombinant protein. In some embodiments, CNS-targeting molecules may be utilized to enable a small molecule, an antibody, scFV, ASO (antisense oligonucleotide), siRNA, lipid, polymer or recombinant protein to cross the blood brain barrier.

In some embodiments, the sequences are part of an engineered AAV capsid protein. In some embodiments, the engineered AAV capsid protein is any engineered AAV capsid protein disclosed herein.

In some embodiments, the sequences may increase tropism of an AAV capsid protein to a cell or tissue of the CNS. In some embodiments, the cell of the CNS is a neuron (e.g., excitatory, inhibitory, motor, sensory, autonomic, sympathetic, parasympathetic, Purkinje, Betz, etc.), a glial cells (e.g., microglia, astrocytes, oligodendrocytes) and/or a supporting cells of the brain such as immune cells (e.g., T cells). In some embodiments, the CNS tissue is the cortex (e.g., frontal, parietal, occipital, temporal), thalamus, hypothalamus, striatum, caudate nucleus, hippocampus putamen, basal ganglia, or entorhinal cortex.

In some embodiments, the sequences increase tropism of an AAV capsid protein to a cell, region, or tissue of the PNS. In some embodiments, the cell or tissue of the PNS is dorsal root ganglion (DRG).

In some embodiments, the sequence comprises at least 4 contiguous amino acids of a sequence set forth in SEQ ID NO: 1-6. In some embodiments, the sequence comprises the sequence set forth in SEQ ID NO: 1-6.

In some embodiments, the sequence comprises variants of the amino acid sequences set forth in SEQ ID NO: 1-6. In embodiments, a variant refers to any one or more of a substitution, deletion, or addition to any of the amino acids of any of the amino acid sequences set forth in SEQ ID NO: 1-6. In some embodiments, the variant comprises 1, 2, 3, or 4 substitutions to any of the amino acids of any of the amino acid sequences set forth in SEQ ID NO: 1-6. In some embodiments, the variant comprises 1, 2, 3, or 4 deletions to any of the amino acids of any of the amino acid sequences set forth in SEQ ID NO: 1-6. In some embodiments, the variant comprises 1, 2, 3, or 4 additions to any of the amino acids of any of the amino acid sequences set forth in SEQ ID NO: 1-6. In some embodiments, the variant comprises any combination of the substitutions, deletions, or additions described above. In some embodiments, the variant amino acid sequences comprise conservative replacement of one or more amino acid(s) within the amino acid sequences set forth in SEQ ID NO: 1-6.

In embodiments, a variant refers to a variant in the nucleotide sequence that encodes any of the amino acid sequences set forth in SEQ ID NO: 1-6. In embodiments, the variant in the nucleotide sequence results in encoding any one or more of a substitution, deletion, or addition to any of the amino acids of any of the amino acid sequences set forth in SEQ ID NO: 1-6. In embodiments, the variant in the nucleotide sequence encodes 1, 2, 3, or 4 substitutions to any of the amino acids of any of the amino acid sequences set forth in SEQ ID NO: 1-6. In embodiments, the variant in the nucleotide sequence encodes 1, 2, 3, or 4 deletions to any of the amino acids of any of the amino acid sequences set forth in SEQ ID NO: 1-6. In embodiments, the variant in the nucleotide sequence encodes 1, 2, 3, or 4 additions to any of the amino acids of any of the amino acid sequences set forth in SEQ ID NO: 1-6. In embodiments, the variant in the nucleotide sequence encodes any combination of the substitutions, deletions, or additions described above.

In some embodiments the capsid protein comprises any of the serotypes of AAV1, AAV2, AAV3B, AAV5, AAV6, AAV8 and AAV9. In embodiments, the capsid protein comprises the serotype AAV2 or AAV9.

In some embodiments, the capsid protein comprises a variant of the STAC-102 parent capsid. In some embodiments, the capsid protein comprises a variant of the sequence set forth in SEQ ID NO: 8. In embodiments, a variant refers to any one or more of a substitution, deletion, or addition to any of the amino acids in the amino acid sequences set forth in SEQ ID NO: 8. In embodiments, the variant comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 substitutions of any of the amino acids of the amino acid sequence of SEQ ID NO: 8. In embodiments, the variant comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 deletions of any of the amino acids of the amino acid sequence set forth of SEQ ID NO: 8. In embodiments, the variant comprises 1, 2, 3, 4, 5, 6, 7, 8, 9, 10, 11, 12, 13, 14, 15, 16, 17, 18, 19, or 20 additions of any of the amino acids of the amino acid sequence set forth SEQ ID NO: 8.

In embodiments, a variant refers to a variant in the nucleotide sequence that encodes the amino acid sequence set forth in SEQ ID NO: 8. In some embodiments, the variant in the nucleotide sequence results in encoding any one or more of a substitution, deletion, or addition to any of the amino acids of the amino acid sequence set forth in SEQ ID NO: 8. In embodiments, the variant in the nucleotide sequence encodes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 substitutions of any of the amino acids of the amino acid sequence of SEQ ID NO: 8. In embodiments, the variant in the nucleotide sequence encodes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 deletions of any of the amino acids of the amino acid sequence of SEQ ID NO: 8. In some embodiments, the variant in the nucleotide sequence encodes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 additions of any of the amino acids of the amino acid sequence of SEQ ID NO: 8.

### Administering AAVs Having Diversified Region Sequences to Subjects

In some embodiments, when administered to subjects, capsid proteins containing the engineered diversified region sequences described herein are capable of higher expression in neurons relative to capsid proteins that lack the engineered diversified region sequences. In some embodiments, the capsid protein administered to subjects comprises an engineered STAC-102 sequence described herein. In some embodiments, the capsid proteins containing the engineered variant sequences (e.g., variant STAC-102) are capable of at least a 2-fold higher expression in neurons relative to capsid proteins that lack the engineered variant sequences (e.g., variant STAC-102). In some embodiments, the capsid proteins containing the engineered variant sequences (e.g., variant STAC-102) are capable of between 5× and 10× higher expression in neurons relative to capsid proteins that lack the engineered variant sequences (e.g., variant STAC-102).

Length and Structure of Diversified Regions (Sequences Modified within Engineered AAV)

The diversified region within an engineered AAV (e.g., STAC-102) variant sequence, may vary in length. In some embodiments, the diversified region is about 3 to about 20 amino acids in length. In some examples, the diversified region is about 8, 9, 10, 11, 12, 13, 14, 15, 16, 17, or 18 amino acids in length. In some embodiments, the diversified region is between about 8-10, 10-12, 12-14, 14-16, or 16-18 amino acids in length. In some embodiments, the diversified region is about 13 amino acids in length.

In some embodiments, a diversified region may comprise an amino acid sequence of any one SEQ ID NO: 1-6, as described in Table 1. In some embodiments, a diversified region may comprise 4 contiguous amino acid sequences of any of SEQ ID NO: 1-6, as described in Table 1. In some embodiments, the peptide is isolated, e.g., recombinant.

In some embodiments, the diversified region comprises at least 3, 4, 5, 6, 7, 8, 9, 10, 11, 12, or 13 consecutive amino acids of any sequence set forth in SEQ ID NO: 1-6. In some embodiments, the diversified region comprises at least 3 contiguous amino acids of any sequence set forth in SEQ ID NO: 1-6 In some embodiments, the diversified region comprises at least 4 contiguous amino acids of any sequence set forth in SEQ ID NO: 1-6. In some embodiments, the diversified region comprises at least 5 contiguous amino acids of any sequence set forth in SEQ ID NO: 1-6. In some embodiments, the diversified region comprises at least 6 contiguous amino acids of any sequence set forth in SEQ ID NO: 1-6. In some embodiments, the diversified region comprises at least 7 contiguous amino acids of any sequence set forth in SEQ ID NO: 1-6. In some embodiments, the diversified region comprises at least 8 contiguous amino acids of any sequence set forth in SEQ ID NO: 1-6.

In some embodiments, the diversified region comprises at least 80%, at least 81%, at least 82%, at least 83%, at least 84%, at least 85%, at least 86%, at least 87%, at least 88%, at least 89%, at least 90%, at least 91%, at least 92% percent, at least 93%, at least 94%, at least 95%, at least 96%, at least 97%, at least 98%, or at least 99% sequence identity with any of SEQ ID NO: 1-6. In some embodiments, the peptide comprises any of SEQ ID NO: 1-6.

In some embodiments, the diversified region comprises the amino acid sequence of SEQ ID NO: 1. In some embodiments, the diversified region comprises an amino acid sequence comprises at least one, two, or three modifications, e.g., substitutions (e.g., conservative substitutions), insertions, or deletions, relative to the amino acid sequence of SEQ ID NO: 1. In some embodiments, the amino acid sequence comprises at least one, two, or three different amino acids relative to the amino acid sequence of SEQ ID NO: 1.

In some embodiments, SEQ ID NO: 1 comprises 13 amino acids with each amino acid comprising consecutive amino acid positions, N1, N2, N3, N4, N5, N6, N7, N8, N9, N10, N11, N12, and N13. In some embodiments, position N5 in SEQ ID NO: 1 is modified. In some embodiments, the modification at position N5 comprises a substitution. In some embodiments, position N8 in SEQ ID NO: 1 is modified. In some embodiments, the modification at position N8 comprises a substitution. In some embodiments, both positions N5 and N8 are modified in SEQ ID NO: 1. In some embodiments, the modifications at positions N5 and N8, comprise a substitution at both N5 and N8 positions.

In some embodiments, position N5 of SEQ ID NO: 1 or a variant of SEQ ID NO: 1 comprises the amino acid K. In some embodiments, position N5 comprises the amino acid M. In some embodiments, position N5 comprises the amino acid N. In some embodiments, position N5 comprises the amino acid Y. In some embodiments, position N5 comprises the amino acid P. In some embodiments, position N8 comprises the amino acid M. In some embodiments, position N8 comprises the amino acid H. In some embodiments, position N8 comprises the amino acid N. In some embodiments, position N8 comprises the amino acid S. In some embodiments, position N8 comprises the amino acid T. In some embodiments, position N8 comprises the amino acid A. In some embodiments, position N8 comprises the amino acid I. In some embodiments, position N8 comprises the amino acid L. In some embodiments, position N8 comprises the amino acid F. In some embodiments, position N8 comprises the amino acid Y. In some embodiments, position N8 comprises the amino acid P. In some embodiments, position N5 and position N8 comprise any combination of the foregoing.

In some embodiments, the diversified region comprises the amino acid sequence of SEQ ID NO: 2. In some embodiments, the diversified region comprises an amino acid sequence comprises at least one, two, or three, e.g., substitutions (e.g., conservative substitutions), insertions, or deletions, relative to the amino acid sequence of SEQ ID NO: 2. In some embodiments, the amino acid sequence comprises at least one, two, or three different amino acids relative to the amino acid sequence of SEQ ID NO: 2.

In some embodiments, SEQ ID NO: 2 comprises 13 amino acids with each amino acid comprising consecutive amino acid positions, N1, N2, N3, N4, N5, N6, N7, N8, N9, N10, N11, N12, and N13. In some embodiments, position N7 in SEQ ID NO: 2 is modified. In some embodiments, the modification at position N7 comprises a substitution. In some embodiments, position N8 in SEQ ID NO: 2 is modified. In some embodiments, the modification at position N8 comprises a substitution. In some embodiments, both positions N7 and N8 are modified in SEQ ID NO: 2. In some embodiments, the modifications at positions N7 and N8, comprise a substitution at both N7 and N8 positions.

In some embodiments, position N7 of SEQ ID NO: 2 or a variant of SEQ ID NO: 2 comprises the amino acid K. In some embodiments, position N7 comprises the amino acid M. In some embodiments, position N7 comprises the amino acid N. In some embodiments, position N7 comprises the amino acid L. In some embodiments, position N8 comprises the amino acid S. In some embodiments, position N8 comprises the amino acid Q. In some embodiments, position N8 comprises the amino acid M. In some embodiments, position N7 and position N8 comprise any combination of the foregoing.

In some embodiments, the diversified region comprises the amino acid sequence of SEQ ID NO: 3. In some embodiments, the diversified region comprises an amino acid sequence comprises at least one, two, or three modifications, e.g., substitutions (e.g., conservative substitutions), insertions, or deletions, relative to the amino acid sequence of SEQ ID NO: 3. In some embodiments, the amino acid sequence comprises at least one, two, or three different amino acids relative to the amino acid sequence of SEQ ID NO: 3.

In some embodiments, SEQ ID NO: 3 comprises 13 amino acids with each amino acid comprising consecutive amino acid positions, N1, N2, N3, N4, N5, N6, N7, N8, N9, N10, N11, N12, and N13. In some embodiments, position N4 in SEQ ID NO: 3 is modified. In some embodiments, the modification at position N4 comprises a substitution. In some embodiments, position N9 in SEQ ID NO: 3 is modified. In some embodiments, the modification at position N9 comprises a substitution. In some embodiments, both positions N4 and N9 are modified in SEQ ID NO: 3. In some embodiments, the modifications at positions N4 and N9, comprise a substitution at both N4 and N9 positions.

In some embodiments, position N4 of SEQ ID NO: 3 or a variant of SEQ ID NO: 3 comprises the amino acid K. In some embodiments, position N4 comprises the amino acid R. In some embodiments, position N4 comprises the amino acid F. In some embodiments, position N4 comprises the amino acid Y. In some embodiments, position N4 comprises the amino acid G. In some embodiments, position N9 comprises the amino acid D. In some embodiments, position N9 comprises the amino acid K. In some embodiments, position N9 comprises the amino acid N. In some embodiments, position N9 comprises the amino acid S. In some embodiments, position N4 and position N9 comprise any combination of the foregoing.

In some embodiments, the diversified region comprises the amino acid sequence of SEQ ID NO: 4. In some embodiments, the diversified region comprises an amino acid sequence comprising at least one, two, or three modifications, e.g., substitutions (e.g., conservative substitutions), insertions, or deletions, relative to the amino acid sequence of SEQ ID NO: 4. In some embodiments, the amino acid sequence comprises at least one, two, or three different amino acids relative to the amino acid sequence of SEQ ID NO: 4.

In some embodiments, SEQ ID NO: 4 comprises 13 amino acids with each amino acid comprising consecutive amino acid positions, N1, N2, N3, N4, N5, N6, N7, N8, N9, N10, N11, N12, and N13. In some embodiments, position N11 in SEQ ID NO: 4 is modified. In some embodiments, the modification at position N11 comprises a substitution. In some embodiments, position N12 in SEQ ID NO: 4 is modified. In some embodiments, the modification at position N12 comprises a substitution. In some embodiments, both positions N11 and N12 are modified in SEQ ID NO: 4. In some embodiments, the modifications at positions N11 and N12, comprise a substitution at both N11 and N12 positions.

In some embodiments, position N11 of SEQ ID NO: 4 or a variant of SEQ ID NO: 4 comprises the amino acid K. In some embodiments, position N11 comprises the amino acid S. In some embodiments, position N11 comprises the amino acid L. In some embodiments, position N11 comprises the amino acid Y. In some embodiments, position N11 comprises the amino acid G. In some embodiments, position N11 comprises the amino acid P. In some embodiments, position N12 comprises the amino acid T. In some embodiments, position N12 comprises the amino acid D. In some embodiments, position N12 comprises the amino acid E. In some embodiments, position N12 comprises the amino acid V. In some embodiments, position N11 and position N12 comprise any combination of the foregoing.

In some embodiments, the diversified region comprises the amino acid sequence of SEQ ID NO: 5. In some embodiments, the amino acid sequence comprises at least one, two, or three modifications, e.g., substitutions (e.g., conservative substitutions), insertions, or deletions, relative to the amino acid sequence of SEQ ID NO: 5. In some embodiments, the amino acid sequence comprises at least one, two, or three different amino acids relative to the amino acid sequence of SEQ ID NO: 5.

In some embodiments, SEQ ID NO: 5 comprises 13 amino acids with each amino acid comprising consecutive amino acid positions, N1, N2, N3, N4, N5, N6, N7, N8, N9, N10, N11, N12, and N13. In some embodiments, position N4 in SEQ ID NO: 5 is modified. In some embodiments, the modification at position N4 comprises a substitution. In some embodiments, position N7 in SEQ ID NO: 5 is modified. In some embodiments, the modification at position N7 comprises a substitution. In some embodiments, both positions N4 and N7 are modified in SEQ ID NO: 5. In some embodiments, the modifications at positions N4 and N7, comprise a substitution at both N4 and N7 positions.

In some embodiments, position N4 of SEQ ID NO: 5 or a variant of SEQ ID NO: 5 comprises the amino acid T. In some embodiments, position N4 comprises the amino acid M. In some embodiments, position N4 comprises the amino acid D. In some embodiments, position N4 comprises the amino acid E. In some embodiments, position N4 comprises the amino acid E. In some embodiments, position N4 comprises the amino acid Q. In some embodiments, position N4 comprises the amino acid S. In some embodiments, position N4 comprises the amino acid V. In some embodiments, position N4 comprises the amino acid G. In some embodiments, position N7 comprises the amino acid P. In some embodiments, position N7 comprises the amino acid H. In some embodiments, position N7 comprises the amino acid R. In some embodiments, position N7 comprises the amino acid A. In some embodiments, position N7 comprises the amino acid V. In some embodiments, position N14 and position N17 comprise any combination of the foregoing.

In some embodiments, the diversified region comprises the amino acid sequence of SEQ ID NO: 6, an amino acid sequence comprising at least one, two, or three modifications, e.g., substitutions (e.g., conservative substitutions), insertions, or deletions, relative to the amino acid sequence of SEQ ID NO: 6. In some embodiments, the amino acid sequence comprises at least one, two, or three different amino acids relative to the amino acid sequence of SEQ ID NO: 6.

In some embodiments, SEQ ID NO: 6 comprises 13 amino acids with each amino acid comprising consecutive amino acid positions, N1, N2, N3, N4, N5, N6, N7, N8, N9, N10, N11, N12, and N13. In some embodiments, position N3 in SEQ ID NO: 6 is modified. In some embodiments, the modification at position N3 comprises a substitution. In some embodiments, position N4 in SEQ ID NO: 6 is modified. In some embodiments, the modification at position N4 comprises a substitution. In some embodiments, both positions N3 and N4 are modified in SEQ ID NO: 6. In some embodiments, the modifications at positions N3 and N4, comprise a substitution at both N3 and N4 positions.

In some embodiments, position N3 of SEQ ID NO: 6 or a variant of SEQ ID NO: 6 comprises the amino acid L. In some embodiments, position N3 comprises the amino acid E. In some embodiments, position N3 comprises the amino acid R. In some embodiments, position N3 comprises the amino acid V. In some embodiments, position N4 comprises the amino acid T. In some embodiments, position N4 comprises the amino acid H. In some embodiments, position N4 comprises the amino acid E. In some embodiments, position N4 comprises the amino acid A. In some embodiments, position N4 comprises the amino acid P. In some embodiments, position N3 and position N4 comprise any combination of the foregoing.

### Functional Properties of Engineered Variant AAV Sequences

In some embodiments, an engineered variant AAV sequence comprising a diversified region sequence as described herein (e.g., variant STAC-102) is used for enhanced or improved transduction of a target cell or tissue (e.g., cells or tissues of the central nervous system (CNS) or peripheral nervous system (PNS)). In some embodiments, the diversified region is used to facilitate the AAV capsid protein across the blood brain barrier following administration to a subject. In some embodiments, the diversified region is used for enhanced or improved distribution of the genetic material throughout the multiple brain regions, e.g., frontal cortex, sensory cortex, motor cortex, putamen, thalamus, cerebellar cortex, dentate nucleus, caudate, and/or hippocampus. In some embodiments, the diversified region is used for enhanced or improved genetic material expression in multiple brain regions. In some embodiments, the diversified region is used for enhanced or improved delivery genetic material of interest to a desired tissue, cell, or organelle.

In some embodiments, the diversified region is included in an engineered AAV capsid proteins and increases tropism of the AAV capsid protein to a cell, region, or tissue of the CNS. Examples of CNS cells include but are not limited to neurons (e.g., excitatory neurons, inhibitory neurons, and motor neurons) and glial cells (e.g., ependymal cells, astrocytes, oligodendrocytes. Examples of CNS tissue include but are not limited to the cortex (e.g., frontal cortex, parietal cortex, occipital cortex, temporal cortex), thalamus, hypothalamus, striatum, hippocampus, entorhinal cortex, and basal ganglia.

In some embodiments, the AAV capsid protein containing a diversified region described herein is capable of increased expression of the AAV capsid protein of at least 0.08-fold in a specific cell, region, or tissue, relative to an AAV capsid protein that lacks a targeting diversified region. In some embodiments, the AAV capsid protein containing a diversified region is capable of increased expression between 0.08-fold and 10-fold relative to an AAV capsid protein that lacks a diversified region, for example, between 0.08-fold and 2-fold, between 2-fold and 3-fold, between 4-fold and 5-fold, between 5-fold and 6-fold, between 6-fold and 7-fold, between 7-fold and 8-fold, between 8-fold and 9-fold, or between 9-fold or 10-fold. In some embodiments, the AAV capsid protein containing a diversified region is capable of increased expression of the AAV capsid protein of greater than 10-fold in a specific cell, region, or tissue, relative to an AAV capsid protein that lacks a diversified region.

### Genetic Material

In some embodiments, the engineered AAV capsid proteins described herein encapsulate genetic material to be delivered to a cell of interest. As such, in embodiments the engineered AAV capsid proteins described herein enable delivery of genetic material to a cell of interest. In embodiments, the genetic material encodes a zinc finger protein, a TALE protein, and/or a CRISPR protein, or fragments thereof. In embodiments, the genetic material encodes one or more antibodies or an antibody fragments. In some embodiments the genetic material encodes one or more regulatory RNA, such as RNAi agents or microRNA.

In some embodiments, the genetic material can include sequences that are coding sequences. In some embodiments, the genetic material can include sequences that are non-coding sequences. In some embodiments, the genetic material can include sequences that are both coding sequences and non-coding sequences. In some embodiments, the expression of the genetic material is capable of being regulated. In some embodiments, the genetic material comprises elements that are regulatable.

In some embodiments, mRNA is encoded in the genetic material. In some embodiments, the mRNA is codon optimized.

In some embodiments, the genetic material encodes a gene therapy product. A gene therapy product can include a peptide, a polypeptide, or an RNA molecule that when expressed carries out a desired therapeutic effect. In some embodiments, the therapeutic effect is treating any one or more diseases or disorders described herein.

In some embodiments, a promoter is operably linked to the genetic material to be delivered to the cell. In some embodiments, the promoter comprises a tissue and/or cell specific promoter. In some embodiments, the one more promoters comprise a ubiquitous promoter. Examples of ubiquitous promoters include cytomegalovirus (CMV), chicken β-actin (CBA), ubiquitin C (UBC), and elongation factor 1α-subunit (EF1-α), amongst others. In some embodiments, the promoter comprises a cell type and/or tissue specific type promoter. Exemplary cell type and/or tissue specific promoters include the human synapsin promoter (hSynl), only expressed in neurons, or the transthyretin promoter (TTR), expressed in hepatocytes. Other non-limiting cell type and/or tissue specific promoters for use in the methods and compositions of the invention include cytokeratin 18 and 19 (epithelial cell specific, Other cell-specific promoters include GFAP promoter (astrocytes), TBG promoter (liver), CAMK promoter (skeletal muscle), MYH6 promoter (cardiomyocytes). In embodiments, tissue specific or cell specific promoters can restrict expression to tissues or cells of the CNS or PNS. In embodiments, tissue specific or cell specific promoters can be used to restrict expression to neurons of the sympathetic system, the parasympathetic system, astrocytes, microglia, oligodendrocytes, and/or Schwann cells.

In some embodiments, the promoters are naturally occurring promoters. In some embodiments, the promoter is synthetic. In some embodiments, the promoter is derived from mammals, humans, viruses, or plants. In some embodiments, the promoters are truncated. In some embodiments, the promoter is mutated.

### Active Agents

In some embodiments, engineered variant AAV sequences described herein (e.g., variant STAC-102) are fused or coupled to an active agent. In some embodiments, a sequence is fused or coupled to an active agent through conjugation. In some embodiments, the active agent comprises a therapeutic agent. In some embodiments, the therapeutic agent comprises an antibody or a portion of an antibody (e.g., Fc region). In some embodiments, the sequence is fused to a Fc region of an antibody. In some embodiments, the sequence is fused to the C-terminus of the Fc region. In some embodiments, the sequence is fused to the N-terminus of the Fc region. In some embodiments, the therapeutic agent comprises an RNAi agent (e.g., siRNA, shRNA, lncRNA, piRNA, snoRNA, or miRNA). In some embodiments, the sequence is fused or coupled directly to at least on strand of the RNAi. In some embodiments, the sequence is fused or coupled to at least one strand of RNAi using a linker. In some embodiments, the sequence is fused or coupled to the sense strand of RNAi. In some embodiments, the sequence is fused or coupled to the antisense strand of RNAi.

In some embodiments the active agent comprises a diagnostic agent. In some embodiments, the diagnostic agent comprises a detectable moiety such as a fluorophore. In some embodiments, the active agent is a small molecule.

### Pharmaceutical Compositions and Dosage Forms

Compositions herein (e.g., engineered STAC-102 variant sequences, AAV particles, and engineered AAV capsid proteins) can be included in pharmaceutical compositions. In some embodiments, the pharmaceutical compositions can include one or more excipients or diluents to (1) increase stability; (2) increase cell transfection or transduction; (3) permit the sustained or delayed release of the genetic material; (4) alter the biodistribution (e.g., target the composition to specific tissues or cell types); (5) increase the translation of encoded protein; (6) alter the release profile of encoded protein and/or (7) allow for regulatable expression of the genetic material.

The pharmaceutical compositions described herein can be administered periodically, such as once or twice a day, or any other suitable time period. For example, pharmaceutical compositions may be administered to a subject in need once a week, once every other week, once every three weeks, once a month, every other month, every three months, every six months, every nine months, once a year, every eighteen months, every two years, every thirty months, or every three years.

In some embodiments, the compositions described herein (e.g., engineered STAC-102 variant sequences, engineered AAV capsid proteins, and modified STAC-102 parent capsids) can be formulated in a wide variety of dosage forms, including but not limited to nasal, pulmonary, oral, topical, or parenteral dosage forms for clinical. Each of the dosage forms can comprise various solubilizing agents, disintegrating agents, surfactants, fillers, thickeners, binders, diluents such as wetting agents or other pharmaceutically acceptable excipients. The compositions described herein can also be formulated for injection, insufflation, infusion, or intradermal exposure. For instance, an injectable formulation may comprise the disclosed compositions in an aqueous or non-aqueous solution at a suitable pH and tonicity. The compositions can be included liquid dosage form for oral administration, such as suspensions, emulsions, or syrups.

In some embodiments, the pharmaceutical compositions described herein function to increase the stability, increase transduction or transfection efficiency, impact biodistribution, increase expression of the protein, and/or alter the release profile.

### Gene Editing System

In some embodiments, the genetic material of interest comprises a gene editing system or portions of a gene editing system. In some embodiments, the gene editing system is capable of inducing single or double-stranded breaks into nucleic acid sequences. In some embodiments, the gene editing system is capable of inserting, substituting, or deleting a base or a sequences of bases into nucleic acid sequences. In some embodiments, the gene editing system includes a CRISPR-Cas system. In some embodiments, the gene editing system includes a TALEN. In some embodiments, the gene editing system includes a zinc finger nuclease.

Engineered AAV Capsid Proteins within a Cell

In some embodiments, engineered AAV capsid proteins are contained within a cell. In some embodiments, the cell is derived from the CNS. In some embodiments, the cell is derived from the PNS. In some embodiments, the cell is derived from the brain. In some embodiments, the cell is derived from the spinal cord. In some embodiments, the cell is derived from any of the frontal cortex, the sensory cortex, the motor cortex, the cerebellar cortex, the cerebral cortex, the brain stem, the hippocampus, or the thalamus, amongst others.

### Engineered AAV Capsid Proteins Delivered to a Target Cell

The engineered AAV capsid proteins may be delivered to one or more of target cells, tissues, organs, or organisms. In some embodiments, the engineered AAV capsid proteins demonstrate enhanced tropism for a target cell type, tissue or organ. As a non-limiting example, the engineered AAV capsid proteins may have enhanced tropism for cells and tissues of the central or peripheral nervous systems, or cells and tissues of a muscle. The engineered AAV capsid proteins may, in addition, or alternatively, have decreased tropism for an undesired target cell-type, tissue or organ. As a non-limiting example, the engineered AAV capsid proteins may have enhanced tropism for B cells, hematopoietic cells, leukocytes, platelets, macrophages, megakaryocytes, monocytes and/or T cells.

### Methods of Detecting Engineered AAV Capsid Proteins

In some embodiments, a method of identifying an engineered AAV capsid protein with a desired characteristic compared to a natural/wild-type AAV serotype is provided comprising: (i) contacting a cell, cell line, or tissue in vitro or in vivo with any one of the libraries of engineered AAV capsid proteins, (ii) allowing the engineered AAV capsid proteins in said library to transduce the cell, cell line, or tissue; (iii) recovering from the cell, cell line, or tissue the AAV variant; and (iv) identifying the engineered AAV capsid protein with the desired characteristic.

In another aspect, disclosed herein are methods for directed evolution of engineered AAV capsid proteins and identification of an engineered AAV capsid protein with a desired characteristic compared to a natural/wild-type AAV serotype. In some embodiments, the steps for directed evolution of engineered AAV capsid proteins to identify engineered AAV capsid proteins with a desired characteristic compared to a natural/wild-type AAV serotype comprise (i) modifying the natural/wild-type AAV serotype to create variant capsids; (ii) packaging of the variant AAVs in producer cells wherein adenovirus helper and AAV rep functions are supplied in trans; (iii) purification of viral capsid library pools; (iv) administration of the pools in vitro or in vivo; (v) recovery of engineered AAV capsid proteins from target tissues or cell lines; (vi) next-generation sequencing to determine the identity of the engineered variant capsid sequences; (vii) repeated rounds of in vitro or in vivo selection where variants are isolated from a target tissue or cell line; and (viii) full evaluation of enriched variants. In some embodiments, the desired characteristic includes enhanced tissue tropism as compared to the natural/wild-type AAV serotype. In some embodiments, the desired characteristic includes enhanced tissue tropism for tissues of the peripheral nervous system as compared to the natural/wild-type AAV serotype. In some embodiments, the desired characteristic includes enhanced tissue tropism of the central nervous system as compared to the natural/wild-type AAV serotype. In some embodiments, modification of the AAV capsid protein results in an AAV capsid protein comprising any of the STAC-102 sequences described herein.

### Methods of Delivery and Treatment

In some embodiments, methods for introducing the compositions described herein (e.g., engineered STAC-102 variant sequences, AAV particles, and engineered AAV capsid proteins) into cells and/or tissues are provided. In some embodiments, the methods comprise introducing into cells and/or tissues any of the compositions described herein in an amount sufficient to modulate, e.g., increase, the production of a target mRNA and/or protein in the cells and/or tissues.

In some embodiments, the compositions described herein are delivered via a localized delivery route. In some embodiments, the localized delivery route includes any one or more of intramuscular administration, intraparenchymal administration, and intracerebral administration, amongst others. In some embodiments, the compositions described herein are administered via a localized delivery route through a bolus infusion.

In some embodiments, the compositions described herein are administered through systemic administration. In some embodiments, systemic administration includes intravenous administration. In some embodiments, intravenous administration includes subcutaneous administration. In some embodiments, the systemic administration includes intraventricular administration.

In some embodiments, the compositions described herein are administered to the central nervous system of via intraventricular administration and/or intravenous administration. In some embodiments, the compositions described herein are administered to the central nervous system via systemic administration. In some embodiments, the systemic administration is intravenous (IV) injection. In some embodiments, the compositions described herein are administered to the central nervous system via intraventricular administration.

In some embodiments the compositions can be delivered to target cell or target tissue including, but not limited to, the CNS, heart, lung, trachea, esophagus, muscle, bone, cartilage, stomach, pancreas, intestine, liver, bladder, kidney, ureter, urethra, uterus, fallopian tube, ovary, testes, prostate, eye, blood, lymph, or oral mucosa. In some embodiments, the target cell or tissue includes, but is not limited to CNS, heart, lung, trachea, esophagus, muscle, bone, cartilage, stomach, pancreas, intestine, liver, bladder, kidney, ureter, urethra, uterus, fallopian tube, ovary, testes, prostate, eye, blood, lymph, or oral mucosa. In some embodiments, the target cell or target tissue is a CNS cell or tissue. In some embodiments, the target cell or tissue is liver cell or tissue.

In some embodiments, the target cell includes, but is not limited to, neurons, glial cells, astrocytes, oligodendroglia, microglia, Schwann cells, ependymal cells, hepatocytes, stellate fat storing cells, Kupffer cells, liver endothelial cells, epithelial cells, cardiomyocytes, smooth muscle cells, T-cells, B cells, hematopoietic stem cells, and embryonic stem cells.

In some embodiments, the compositions described herein are delivered to the central nervous system through the cerebral spinal fluid pathway. In some embodiments, compositions described herein are administered to the central nervous system via intraparenchymal delivery. In some embodiments, the compositions described herein are administered to the central nervous system via intracranial delivery. In some embodiments, the compositions described herein are delivered to the central nervous system via intraocular delivery. In some embodiments, the compositions described herein are administered to the brain. In some embodiments, the compositions described herein are administered to the brain via injection into the brain. In some embodiments, the compositions described herein are administered to the brain via intrahippocampal injection.

In some embodiments, the compositions described herein are administered as part of a composition that allows for extended release. In some embodiments, the compositions comprises a formulation that includes a depot.

Disclosed herein are methods of treatment using any of the compositions described herein (engineered STAC-102 variant sequences, AAV particles, and engineered AAV capsid proteins). In embodiments, the disclosed compositions can be used to treat any one or more of muscular or neuromuscular disorders, neurooncological disorders, neurological diseases/disorders, and neurodegenerative disorders, amongst others. In embodiments, the disclosed compositions can be used to treat any one or more of Alzheimer's disease, Huntington's disease; autism; Parkinson's disease; Spinal muscular atrophy, Friedreich's ataxia. In embodiments, the disclosed compositions are used in treatments through any of the methods of delivery described herein.

In some embodiments, disclosed are methods for treating, or ameliorating a disease or condition associated with abnormal gene and/or protein in a subject in need of treatment, the methods comprising administering to the subject any effective amount of at least one of the compositions described herein (e.g., engineered STAC-102 variant sequences, AAV particles, and engineered AAV capsid proteins), delivering the compositions described herein into targeted cells, inhibiting or activating the gene expression and protein production, and ameliorating symptoms of the disease or condition in the subject.

### EQUIVALENTS AND SCOPE

The disclosure includes many equivalents to the specific embodiments described herein. A person of skill in the art will be able to ascertain equivalents to the specific embodiments, through routine experimentation.

It is assumed that words of this disclosure are for the purpose of description and not limitation. Changes to words in the claims can be made, while still retaining the scope of the disclosure in its broad aspect. Specific embodiments of the disclosure have been described herein. However, these embodiments are not intended to be limiting of the broad scope of this disclosure.

The disclosure is further illustrated by the following examples, which are intended to be purely illustrative and not limiting of the disclosure herein.

### EXAMPLES

### Example 1. Methods

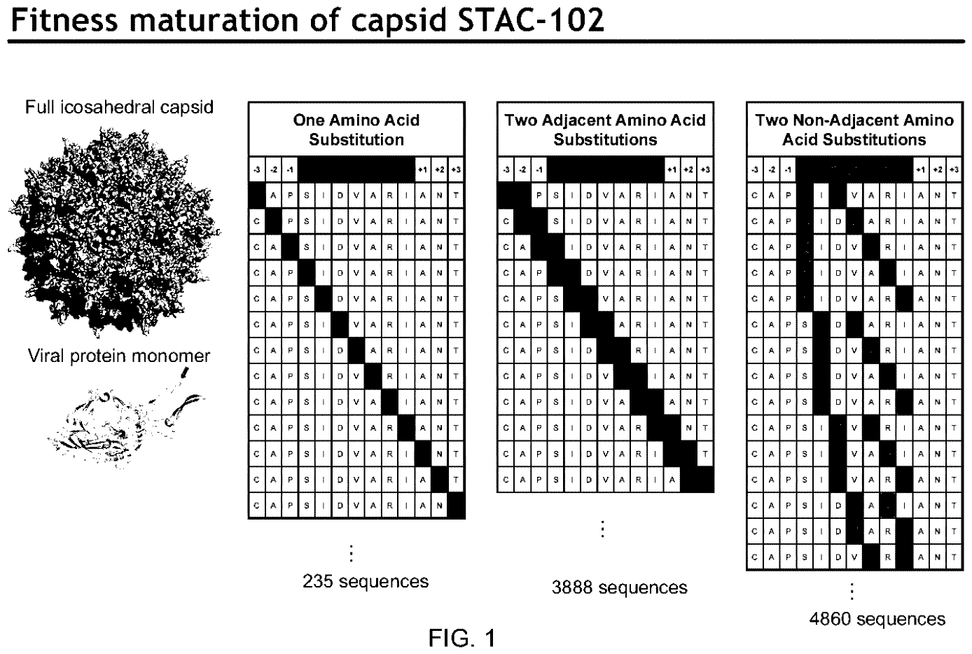

Fitness maturation of AAV capsid STAC-102. Fitness maturation was performed by creating libraries of AAV capsids with 1 or 2 amino acid mutations introduced relative to the parental capsid STAC-102. In FIG. 1 the amino acids 586, 587, and 588 are the three amino acids 5′ of the inserted 7mer peptide MTLTRQE in STAC-102 while amino acids 589, 590, and 591 are immediately 3′ of the peptide. Each mutated position was changed to all possible amino acids except cysteine and the original amino acid in STAC-102.

AAV capsid library generation. Capsid variants for library screening were synthesized as an oligo pool. Each capsid peptide was synthesized with three unique nucleotide sequences encoding the peptide, and each peptide was linked to a distinct barcode. The oligo pool was cloned into a linearized intermediate plasmid, followed by cloning of a constant donor sequence to separate the barcode and peptide region and generate the full AAV vector construct. The resulting construct includes a neuron specific promoter driving expression of the barcodes, wherein each barcode is linked to the identity of a single capsid. This enables an assessment of which capsids drive the most functional mRNA expression in neurons. Moreover, each barcode is linked to a unique molecular identifier (UMI). Based on the overall size of the cloned library each barcode is appended to hundreds to thousands of UMIs. The utility of the UMI is to additionally assess how many distinct AAV transduction events give rise to the NGS read counts that are measured.

These AAV plasmid libraries were manufactured in HEK293 cells. Briefly, libraries were produced by transient transfection including supplementation of Rep in trans, capsids were purified by cesium density centrifugation, and buffer exchanged into PBS by Amicon filtration. DNase-resistant viral genomic titers were measured by quantitative real time PCR.

Administration of AAV libraries to cynomolgus macaques. The AAV capsid library was administered to cynomolgus macaques as a slow bolus injection into the cisterna magna. 1 mL of library test article was administered at rate of 1 mL/min.

Tissue collection and preservation. Two weeks post-administration animals were euthanized and a necropsy was performed. The brain was removed and placed in a coronal brain matrix in an ice-cold nuclease free PBS bath for approximately 10 minutes. The brain was then sliced using a brain matrix at 4 mm coronal slice thickness. Each brain slice was immersed in RNAlater (ratio 1:10) and refrigerated at 4° C. for 24 hrs. Following removal from RNAlater, slices were rinsed briefly in cold RNAse-free phosphate-buffered saline and 2 millimeter punches were collected from different brain regions. Punches and remaining tissue slices were frozen at ≤−65° C. The same workflow was used to process spinal cord and dorsal root ganglia tissues.

Tissue lysis for RNA isolation. Brain and spinal cord punches to be processed were placed on dry ice. Approximately one half of each tissue sample was excised and transferred to an Eppendorf tube pre-filled with 600 μL of TRI Reagent and 2, 3.2 mm steel beads. Sample tubes were placed into a Retsch MM300 Tissue-Lyser and 6 rounds of homogenization were performed at a frequency of 25.1 Hz for 1½ minutes with a 1-minute pause between rounds to prevent overheating.

RNA isolation from brain punches, spinal cord, and dorsal root ganglia. Isolation and purification of total RNA from homogenized tissue punches preserved in RNAlater was performed using the MagMAX96 Total RNA Isolation Kit in conjunction with the KingFisher Flex Purification System according to the manufacturers protocol. Briefly, bromochloropropane (BCP) was added followed by centrifugation to separate the homogenate into aqueous and organic phases. The aqueous phase, containing partially purified RNA was then transferred to the wells of a 96-well KingFisher processing plate. Isopropanol (100%) was added to each well followed by addition of magnetic RNA binding beads. Subsequent processing was performed on the KingFisherFlex Purification System. Briefly, the RNA binding beads were magnetically captured, and an on-bead DNase digestion and several washes were performed. Purified RNA was eluted in 100 μL of low salt Elution Buffer. The KingFisher processing plate was then transferred to a magnetic stand on the benchtop. The eluants (~90 μL) were transferred away from any residual beads into a 96-well PCR plate for downstream processing. The yield and purity of the RNA was determined using a NanoDrop 8000 spectrophotometer.

RNA isolation from entire coronal brain slices. Hemisected brain slices were weighed and placed into 50 mL Bigprep Lysing Matrix D tubes. The tubes were filled with TRIZOL Reagent at 10 mL per gram of tissue. The tissue samples were homogenized at 4.0 meters/second for 30 seconds with 2 minutes pause. The tissue homogenization was repeated 4 times. The lysate was centrifuged at 4,300 g for 5 minutes at 4° C. The clarified lysate was removed and a second round of centrifugation and removal of clarified lysate to a fresh tube was conducted. 5 mL aliquots were further processed by adding 0.2 mL molecular grade chloroform per mL of Trizol. The samples were mixed by inversion and vortexing for 30 seconds then centrifuged at 4,300 g for 30 minutes at 4° C. The aqueous phase, approximately 50% of the lysate volume or ~2.5 mL, was distributed to 2 mL microcentrifuge tubes. 0.5 mL per mL Trizol of molecular grade isopropanol was added to the tubes and mixed by inversion. The samples were incubated for 10 minutes on ice followed by centrifugation at 12,000 g for 10 minutes at 4° C. The RNA pellet was resuspended in 75% ethanol at 1 mL per mL Trizol, vortexed, and centrifuged at 12,000 g for 5 minutes at 4° C. The RNA pellet was air dried for 5 minutes and dissolved in 0.2 mL per mL Trizol with RNAse free DEPC treated water. The yield and purity of the RNA was determined using a spectrophotometer. Samples were spot checked for RNA integrity using the Agilent RNA 6000 Nano kit and Bioanalyzer 2100.

Reverse transcription and next generation sequencing. Isolated RNA was reverse transcribed using a gene-specific primer with the Qiagen QuantiTect Reverse Transcription Kit. The synthesized complementary DNA (cDNA) was then carried forward for next-generation sequencing (NGS).

Primers were designed to amplify the region of interest on the cDNA, and these primers also included sequences that bind the adapters for NGS. Using the Phusion® HotStart Flex DNA polymerase, a PCR was set up to amplify the region of interest of the cDNA or vector genomic DNA and a second PCR was set up to attach the adapters necessary for NGS sample barcoding. These amplicons were carried forward for NGS and analyzed using a custom bioinformatics pipeline.

### Example 2. Results

Fitness maturation of STAC-102 identified second generation variants with 5-10× higher neuronal mRNA expression in cynomolgus macaque after CSF administration. Library assessment suggests that these capsids have a similar or better manufacturing yield compared to STAC-102. Bioinformatic analysis of fold enrichment, coefficient of variation and UMI recovery was used to select the lead capsids. Second generation STAC-102 capsids will be evaluated individually in cynomolgus macaques and are promising candidates to enable therapeutics for CNS indications.

Multiple assessments of the STAC-102 fitness maturation library were conducted across both in vitro and in vivo settings.

In vitro assessment of novel AAV variant performance and manufacturing productivity. The following library data are presented as bubble plots wherein log 2 fold change enrichment of each capsid variant was normalized to its relative abundance in the administered test article. The coefficient of variation represents the consistency of capsid performance across multiple sequencing reactions. The size of the bubble is proportional to the fraction of sequenced samples in which a capsid is found. The number of unique molecular identifiers is represented by the color according to the color legend.

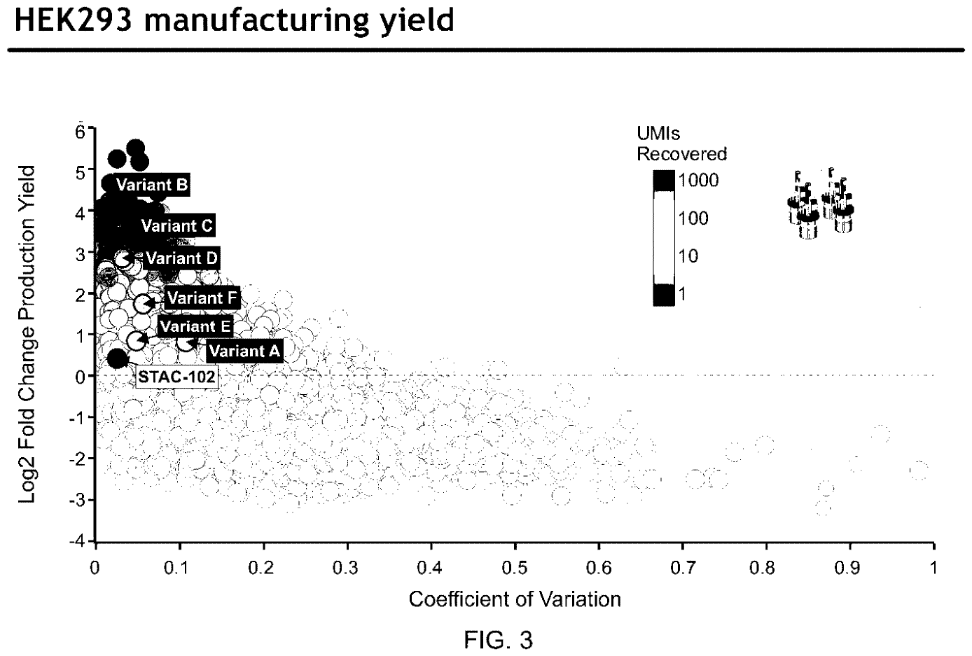

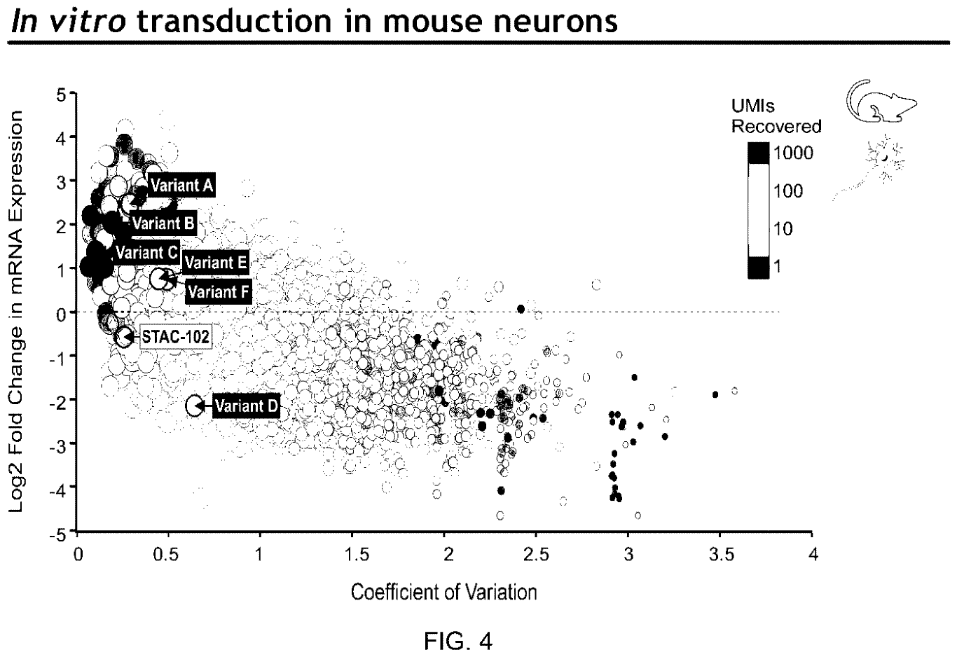

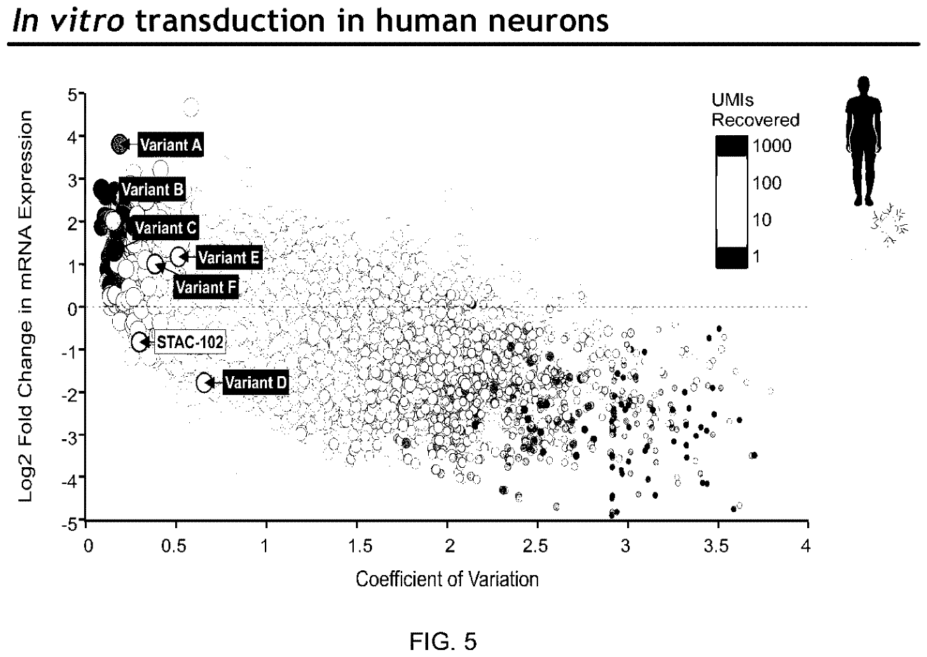

Capsids with favorable performance have the following features in the bubble plots (see FIGS. 3-5):
1) High log 2 fold enrichment, these data are normalized by input abundance (y-axis)
2) Low coefficient of variation (x-axis)
3) High fraction of sequenced samples in which a capsid is found (large bubble size)
4) Robust UMI recovery (green color)

The parent capsid STAC-102 and notable second-generation capsids with improved CNS delivery in cynomolgus macaques are annotated per the following table.

TABLE 1

| Sequence of | | |
|---|---|---|
| diversified region | | |
| (underlined positions | Annotated name of | |
| are mutations | corresponding STAC-102 | Diversified region |
| relative to STAC-102) | variant | SEQ ID NO: |
| RGNMKLTMQERQA | Variant A | 1 |
| RGNMTLKSQERQA | Variant B | 2 |
| RGNKTLTRDERQA | Variant C | 3 |
| RGNMTLTRQEKTA | Variant D | 4 |
| RGNTTLPRQERQA | Variant E | 5 |
| RGLTTLTRQERQA | Variant F | 6 |
| RGNMTLTRQERQA | Diversified Region in | 7 |
| | STAC-102 | |

Conclusions of in vitro assessments. The second generation STAC-102 variants annotated as A-F were selected primarily based on their effective CNS delivery in cynomolgus macaques. Notably, all of these capsids can be manufactured as well or better than the parental STAC-102 sequence. With the exception of Variant D, second generation variants also show improved in vitro potency relative to the parental sequence.

Evaluation of novel AAV performance in cynomolgus macaque brain. Library AAV variant performance was assessed by quantifying enrichment in AAV library mRNA transcript expressed in neurons in macaque CNS tissues.

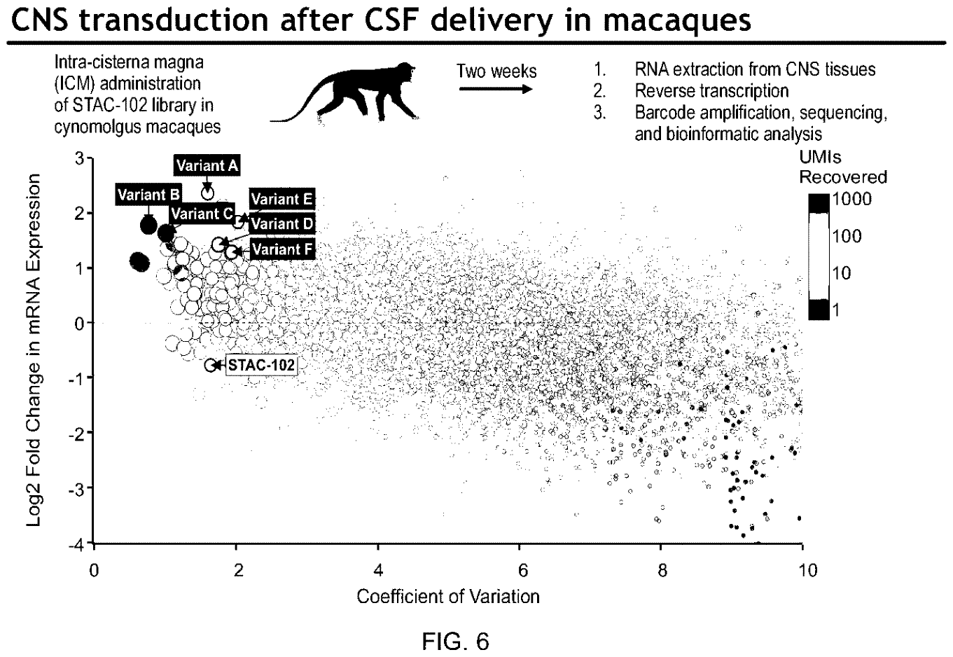

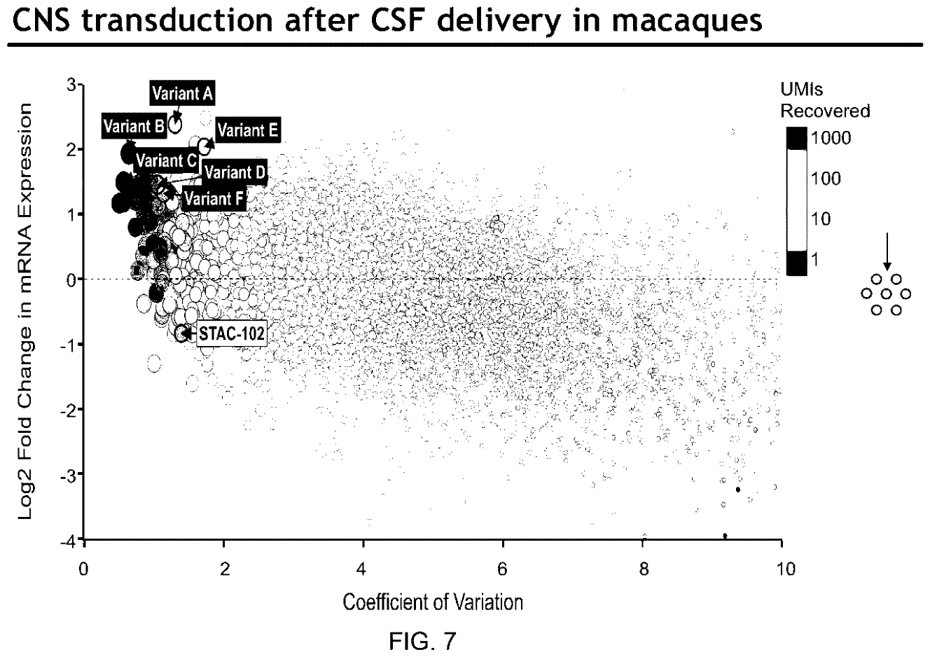

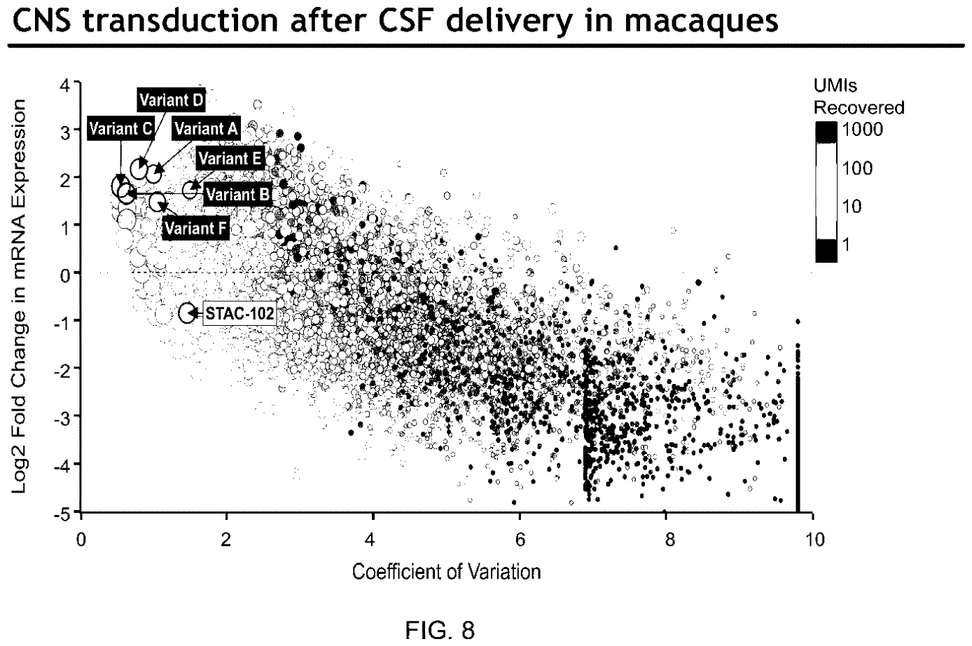

Capsids with favorable performance have the following features in the bubble plots (see FIGS. 6-8):
1) High log 2 fold enrichment, these data are normalized by input abundance (y-axis)
2) Low coefficient of variation (x-axis)
3) High fraction of sequenced samples in which a capsid is found (large bubble size)
4) Robust UMI recovery (green color)

The parent capsid STAC-102 and notable second-generation capsids that perform well for CNS delivery in cynomolgus macaques are annotated.

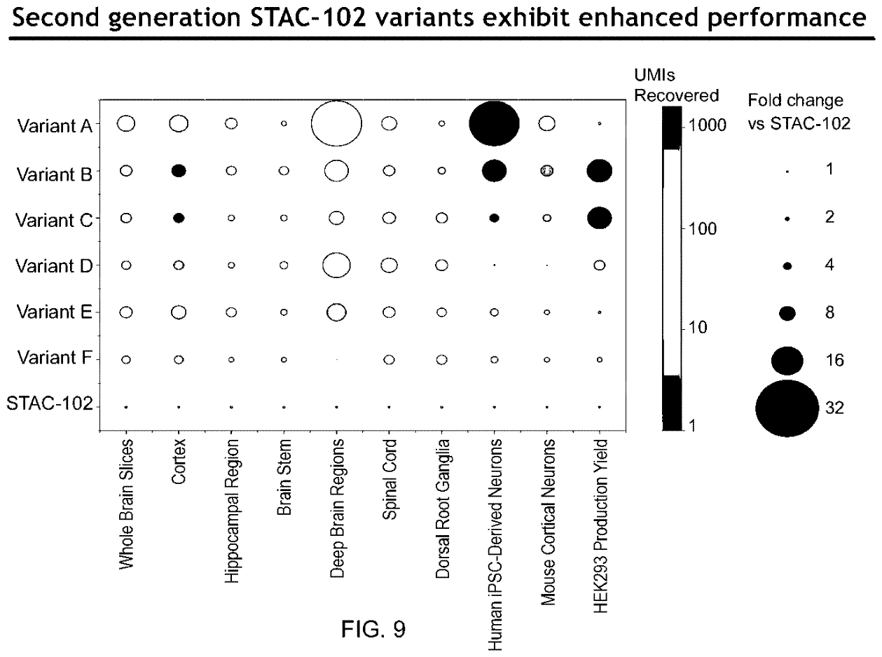

FIG. 9. Summary of second generation STAC-102 capsid performance. Fold change is calculated relative to STAC-102. Bubble size is proportional to fold change and UMI counts are indicated by color. Cortex includes fronto-orbital gyrus, middle frontal gyrus, superior frontal gyrus, anterior cingulate gyrus, posterior cingulate gyrus, inferior frontal gyrus, lateral orbital gyrus, precentral gyrus, superior temporal gyrus, middle temporal gyrus, inferior temporal gyrus, supramarginal gyrus, postcentral gyrus, insula, cuneus, precuneus, lingual gyrus, superior parietal lobule, angular gyrus, occipital gyrus, entorhinal cortex, fusiform gyrus. Hippocampal region includes hippocampus, parahippocampal gyrus, subiculum, amygdala. Deep brain regions include caudate, putamen, substantia nigra, globus pallidus, hypothalamus, thalamus, lateral geniculate nucleus. Brain stem includes midbrain, medulla, pons. Spinal cord includes cervical, thoracic, and lumbar levels. Dorsal root ganglia includes cervical thoracic, and lumbar levels.

Conclusions of in vivo assessment in cynomolgus macaques. The second generation STAC-102 variants annotated as A-F exhibit a substantially higher degree of neuronal mRNA expression in multiple CNS regions compared to the parental STAC-102 sequence.

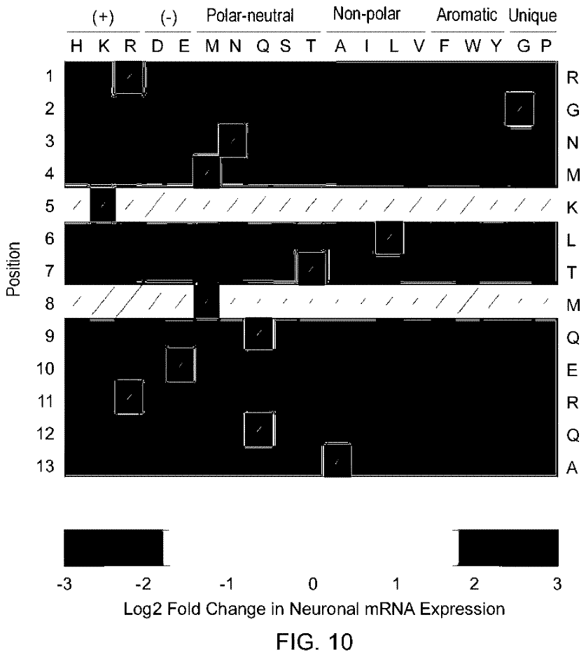

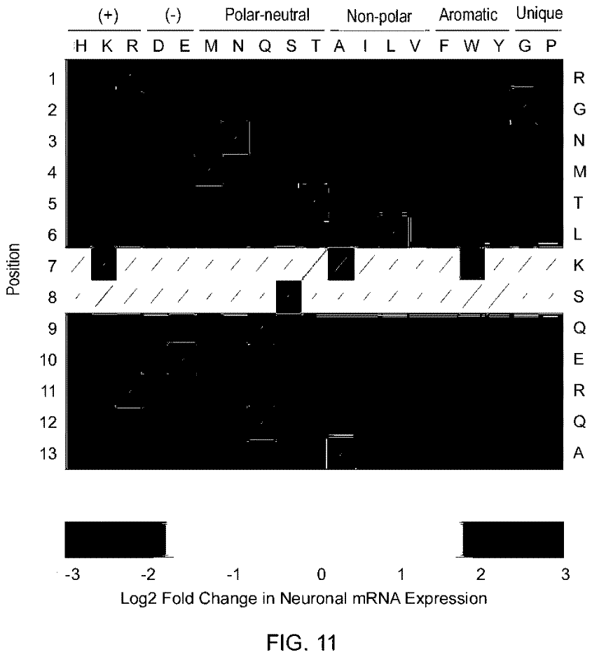

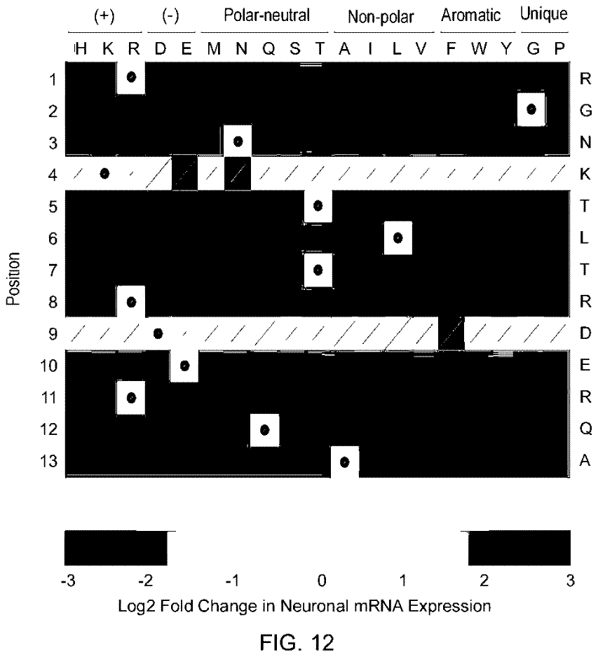

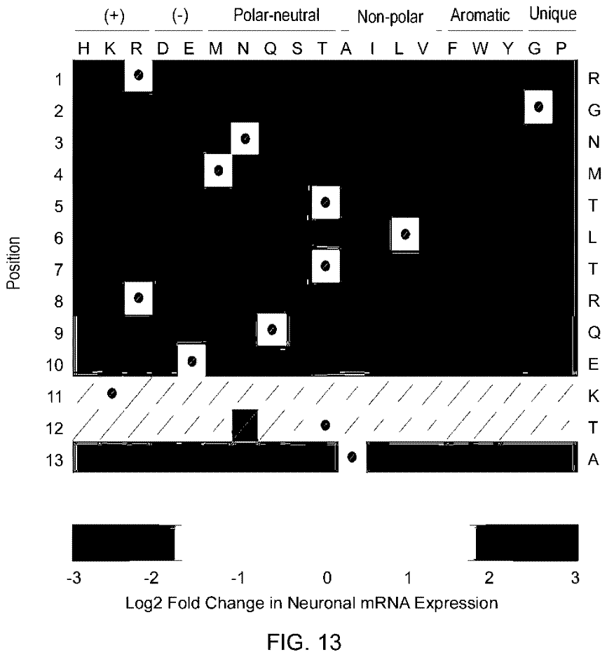

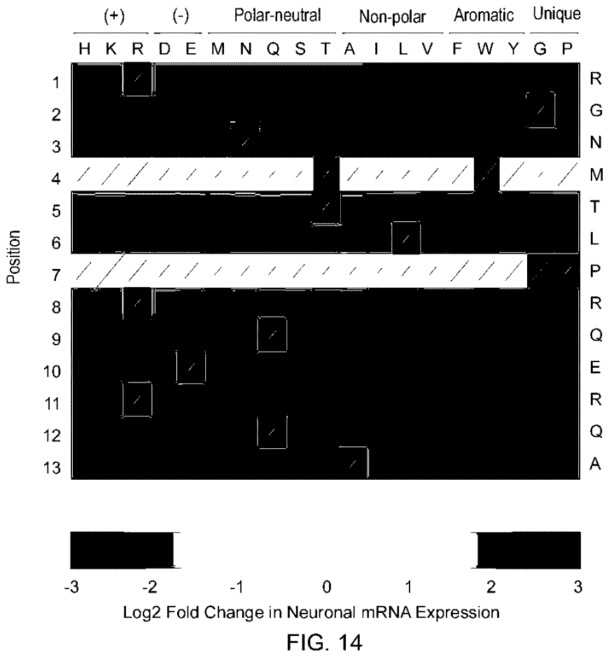

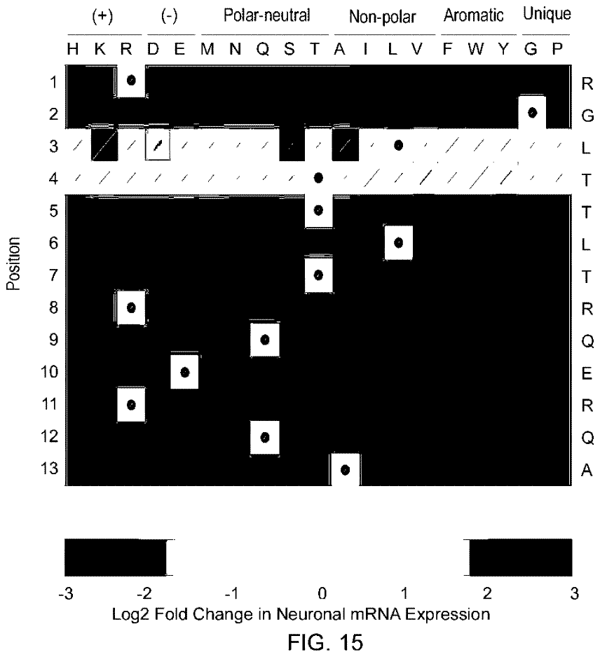

Analysis of mutational hotspots for variants A-F. Based on the library design and fitness maturation approach we were able to additionally assess the performance of many capsids that are closely related to variants A-F. In the heat maps shown in FIGS. 10-15, the squares with black ovals indicate the identity of the amino acid in Variant A at each position, this is also indicated in the amino acid sequence on the right side of the heat map. The diagonal slash is proportional to the coefficient of variation. The top of the heat map shows the different amino acids that were evaluated at each position and the biochemical properties. The color of each square is proportional to the log 2 fold enrichment per the provided color legend. Position refers to the position of each amino acid in the diversified motif. Collectively, the heat maps provide an overview of which mutations at each mutated position are high performing.

Conclusions of mutation analysis. Although variants A-F are the highest performing sequences within each cluster there are several alternative sequences for each capsid that perform at a similar or slightly lower level.

Full capsid amino acid sequences for STAC-102 (SEQ ID NO: 8) and second-generation variants A-F (SEQ ID NO: 9-14). In the sequences below, the diversified region in STAC-102 and its variants are indicated in bold. Mutations relative to STAC-102 are underlined.

STAC-102 Parent Capsid
(SEQ ID NO: 8)
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNMTLTRQERQAATA
DVNTQGVLPGMVWQDRDVYLQGPIWAKIPHTDGHFHPSPLMGGFGLKHPP
PQILIKNTPVPANPSTTFSAAKFASFITQYSTGQVSVEIEWELQKENSKR
WNPEIQYTSNYNKSVNVDFTVDINGVYSEPRPIGTRYLTRNL

### Modified STAC-102 Parent Capsids

Parent Capsid STAC-102 with SEQ ID NO: 1
(SEQ ID NO: 9, Variant A)
MAADGYLPDWLEDTLSEGIRQWWKLKPGPPPPKPAERHKDDSRGLVLPGYKYLGPF
NGLDKGEPVNEADAAALEHDKAYDRQLDSGDNPYLKYNHADAEFQERLKEDTSFG
GNLGRAVFQAKKRVLEPLGLVEEPVKTAPGKKRPVEHSPVEPDSSSGTGKAGQQPA
RKRLNFGQTGDADSVPDPQPLGQPPAAPSGLGTNTMATGSGAPMADNNEGADGVG
NSSGNWHCDSTWMGDRVITTSTRTWALPTYNNHLYKQISSQSGASNDNHYFGYSTP
WGYFDFNRFHCHFSPRDWQRLINNNWGFRPKRLNFKLFNIQVKEVTQNDGTTTIAN
NLTSTVQVFTDSEYQLPYVLGSAHQGCLPPFPADVFMVPQYGYLTLNNGSQAVGRS
SFYCLEYFPSQMLRTGNNFTFSYTFEDVPFHSSYAHSQSLDRLMNPLIDQYLYYLSRT
NTPSGTTTQSRLQFSQAGASDIRDQSRNWLPGPCYRQQRVSKTSADNNNSEYSWTG
ATKYHLNGRDSLVNPGPAMASHKDDEEKFFPQSGVLIFGKQGSEKTNVDIEKVMITD
EEEIRTTNPVATEQYGSVSTNLQRGNMKLTMQERQAATADVNTQGVLPGMVWQD
RDVYLQGPIWAKIPHTDGHFHPSPLMGGFGLKHPPPQILIKNTPVPANPSTTFSAAKF
ASFITQYSTGQVSVEIEWELQKENSKRWNPEIQYTSNYNKSVNVDFTVDTNGVYSEP
RPIGTRYLTRNL
Parent Capsid STAC-102 with SEQ ID NO: 2
(SEQ ID NO: 10, Variant B)
MAADGYLPDWLEDTLSEGIRQWWKLKPGPPPPKPAERHKDDSRGLVLPGYKYLGPF
NGLDKGEPVNEADAAALEHDKAYDRQLDSGDNPYLKYNHADAEFQERLKEDTSFG
GNLGRAVFQAKKRVLEPLGLVEEPVKTAPGKKRPVEHSPVEPDSSSGTGKAGQQPA
RKRLNFGQTGDADSVPDPQPLGQPPAAPSGLGTNTMATGSGAPMADNNEGADGVG
NSSGNWHCDSTWMGDRVITTSTRTWALPTYNNHLYKQISSQSGASNDNHYFGYSTP
WGYFDFNRFHCHFSPRDWQRLINNNWGFRPKRLNFKLFNIQVKEVTQNDGTTTIAN
NLTSTVQVFTDSEYQLPYVLGSAHQGCLPPFPADVFMVPQYGYLTLNNGSQAVGRS
SFYCLEYFPSQMLRTGNNFTFSYTFEDVPFHSSYAHSQSLDRLMNPLIDQYLYYLSRT
NTPSGTTTQSRLQFSQAGASDIRDQSRNWLPGPCYRQQRVSKTSADNNNSEYSWTG
ATKYHLNGRDSLVNPGPAMASHKDDEEKFFPQSGVLIFGKQGSEKTNVDIEKVMITD
EEEIRTTNPVATEQYGSVSTNLQRGNMTLKSQERQAATADVNTQGVLPGMVWQD
RDVYLQGPIWAKIPHTDGHFHPSPLMGGFGLKHPPPQILIKNTPVPANPSTTFSAAKF
ASFITQYSTGQVSVEIEWELQKENSKRWNPEIQYTSNYNKSVNVDFTVDTNGVYSEP
RPIGTRYLTRNL
Parent Capsid STAC-102 with SEQ ID NO: 3
(SEQ ID NO: 11, Variant C)
MAADGYLPDWLEDTLSEGIRQWWKLKPGPPPPKPAERHKDDSRGLVLPGYK
YLGPFNGLDKGEPVNEADAAALEHDKAYDRQLDSGDNPYLKYNHADAEFQERLKE
DTSFGGNLGRAVFQAKKRVLEPLGLVEEPVKTAPGKKRPVEHSPVEPDSSSGTGKAG
QQPARKRLNFGQTGDADSVPDPQPLGQPPAAPSGLGTNTMATGSGAPMADNNEGA
DGVGNSSGNWHCDSTWMGDRVITTSTRTWALPTYNNHLYKQISSQSGASNDNHYF
GYSTPWGYFDFNRFHCHFSPRDWQRLINNNWGFRPKRLNFKLFNIQVKEVTQNDGT
TTIANNLTSTVQVFTDSEYQLPYVLGSAHQGCLPPFPADVFMVPQYGYLTLNNGSQA
VGRSSFYCLEYFPSQMLRTGNNFTFSYTFEDVPFHSSYAHSQSLDRLMNPLIDQYLYY
LSRTNTPSGTTTQSRLQFSQAGASDIRDQSRNWLPGPCYRQQRVSKTSADNNNSEYS
WTGATKYHLNGRDSLVNPGPAMASHKDDEEKFFPQSGVLIFGKQGSEKTNVDIEKV
MITDEEEIRTTNPVATEQYGSVSTNLQRGNKTLTRDERQAATADVNTQGVLPGMV
WQDRDVYLQGPIWAKIPHTDGHFHPSPLMGGFGLKHPPPQILIKNTPVPANPSTTFSA
AKFASFITQYSTGQVSVEIEWELQKENSKRWNPEIQYTSNYNKSVNVDFTVDINGVY
SEPRPIGTRYLTRNL
Parent Capsid STAC-102 with SEQ ID NO: 4
(Variant D, SEQ ID NO: 12)
MAADGYLPDWLEDTLSEGIRQWWKLKPGPPPPKPAERHKDDSRGLVLPGYKYLGPF
NGLDKGEPVNEADAAALEHDKAYDRQLDSGDNPYLKYNHADAEFQERLKEDTSFG
GNLGRAVFQAKKRVLEPLGLVEEPVKTAPGKKRPVEHSPVEPDSSSGTGKAGQQPA
RKRLNFGQTGDADSVPDPQPLGQPPAAPSGLGTNTMATGSGAPMADNNEGADGVG
NSSGNWHCDSTWMGDRVITTSTRTWALPTYNNHLYKQISSQSGASNDNHYFGYSTP
WGYFDFNRFHCHFSPRDWQRLINNNWGFRPKRLNFKLFNIQVKEVTQNDGTTTIAN
NLTSTVQVFTDSEYQLPYVLGSAHQGCLPPFPADVFMVPQYGYLTLNNGSQAVGRS
SFYCLEYFPSQMLRTGNNFTFSYTFEDVPFHSSYAHSQSLDRLMNPLIDQYLYYLSRT
NTPSGTTTQSRLQFSQAGASDIRDQSRNWLPGPCYRQQRVSKTSADNNNSEYSWTG
ATKYHLNGRDSLVNPGPAMASHKDDEEKFFPQSGVLIFGKQGSEKTNVDIEKVMITD
EEEIRTTNPVATEQYGSVSTNLQRGNMTLTRQEKTAATADVNTQGVLPGMVWQD
RDVYLQGPIWAKIPHTDGHFHPSPLMGGFGLKHPPPQILIKNTPVPANPSTTFSAAKF
ASFITQYSTGQVSVEIEWELQKENSKRWNPEIQYTSNYNKSVNVDFTVDINGVYSEP
RPIGTRYLTRNL
Parent Capsid STAC-102 with SEQ ID NO: 5
(SEQ ID NO: 13, Variant E)
MAADGYLPDWLEDTLSEGIRQWWKLKPGPPPPKPAERHKDDSRGLVLPGYKYLGPF
NGLDKGEPVNEADAAALEHDKAYDRQLDSGDNPYLKYNHADAEFQERLKEDTSFG
GNLGRAVFQAKKRVLEPLGLVEEPVKTAPGKKRPVEHSPVEPDSSSGTGKAGQQPA
RKRLNFGQTGDADSVPDPQPLGQPPAAPSGLGTNTMATGSGAPMADNNEGADGVG
NSSGNWHCDSTWMGDRVITTSTRTWALPTYNNHLYKQISSQSGASNDNHYFGYSTP
WGYFDFNRFHCHFSPRDWQRLINNNWGFRPKRLNFKLFNIQVKEVTQNDGTTTIAN
NLTSTVQVFTDSEYQLPYVLGSAHQGCLPPFPADVFMVPQYGYLTLNNGSQAVGRS
SFYCLEYFPSQMLRTGNNFTFSYTFEDVPFHSSYAHSQSLDRLMNPLIDQYLYYLSRT
NTPSGTTTQSRLQFSQAGASDIRDQSRNWLPGPCYRQQRVSKTSADNNNSEYSWTG
ATKYHLNGRDSLVNPGPAMASHKDDEEKFFPQSGVLIFGKQGSEKTNVDIEKVMITD
EEEIRTTNPVATEQYGSVSTNLQRGNTTLPRQERQAATADVNTQGVLPGMVWQDR
DVYLQGPIWAKIPHTDGHFHPSPLMGGFGLKHPPPQILIKNTPVPANPSTTFSAAKFAS
FITQYSTGQVSVEIEWELQKENSKRWNPEIQYTSNYNKSVNVDFTVDTNGVYSEPRPI
GTRYLTRNL
Parent Capsid STAC-102 with SEQ ID NO: 6
(SEQ ID NO: 14, Variant F)
MAADGYLPDWLEDTLSEGIRQWWKLKPGPPPPKPAERHKDDSRGLVLPGYK
YLGPFNGLDKGEPVNEADAAALEHDKAYDRQLDSGDNPYLKYNHADAEFQERLKE
DTSFGGNLGRAVFQAKKRVLEPLGLVEEPVKTAPGKKRPVEHSPVEPDSSSGTGKAG
QQPARKRLNFGQTGDADSVPDPQPLGQPPAAPSGLGTNTMATGSGAPMADNNEGA
DGVGNSSGNWHCDSTWMGDRVITTSTRTWALPTYNNHLYKQISSQSGASNDNHYF
GYSTPWGYFDFNRFHCHFSPRDWQRLINNNWGFRPKRLNFKLFNIQVKEVTQNDGT
TTIANNLTSTVQVFTDSEYQLPYVLGSAHQGCLPPFPADVFMVPQYGYLTLNNGSQA
VGRSSFYCLEYFPSQMLRTGNNFTFSYTFEDVPFHSSYAHSQSLDRLMNPLIDQYLYY
LSRTNTPSGTTTQSRLQFSQAGASDIRDQSRNWLPGPCYRQQRVSKTSADNNNSEYS
WTGATKYHLNGRDSLVNPGPAMASHKDDEEKFFPQSGVLIFGKQGSEKTNVDIEKV
MITDEEEIRTTNPVATEQYGSVSTNLQRGLTTLTRQERQAATADVNTQGVLPGMV
WQDRDVYLQGPIWAKIPHTDGHFHPSPLMGGFGLKHPPPQILIKNTPVPANPSTTFSA
AKFASFITQYSTGQVSVEIEWELQKENSKRWNPEIQYTSNYNKSVNVDFTVDINGVY
SEPRPIGTRYLTRNL

What is claimed is:

1. An engineered AAV capsid protein comprising at least 3, 4, 5, 6, 7, 8, 9, 10, 11, 12, or 13 contiguous amino acids of an amino acid sequence set forth in any one of SEQ ID NO: 1-6.

2. The engineered AAV capsid protein of claim 1, comprising a variant sequence of SEQ ID NO: 1, wherein the variant sequence comprises (i) any of amino acids K, M, N, Y, P at amino acid position 5 of SEQ ID NO: 1 and/or (ii) any of amino acids M, H, N, S, T, A, I, L, F, Y, and P at amino acid position 8 of SEQ ID NO: 1; optionally wherein the AAV capsid protein comprises SEQ ID NO: 1.

3. The engineered AAV capsid protein of claim 1, comprising a variant sequence of SEQ ID NO: 2, wherein the variant sequence comprises (i) any of amino acids K, M, N, and L at amino acid position 7 of SEQ ID NO: 2 and/or (ii) any of amino acids S, Q, and M at amino acid position 8 of SEQ ID NO: 2; optionally wherein the AAV capsid protein comprises SEQ ID NO: 2.

4. The engineered AAV capsid protein of claim 1, comprising a variant sequence of SEQ ID NO: 3, wherein the variant sequence comprises (i) any of amino acids K, R, F, Y, and G at amino acid position 4 of SEQ ID NO: 3 and/or (ii) any of amino acids D, K, N, and S at amino acid position 9 of SEQ ID NO: 3; optionally wherein the AAV capsid protein comprises SEQ ID NO: 3.

5. The engineered AAV capsid protein of claim 1, comprising a variant sequence of SEQ ID NO: 4, wherein the variant sequence comprises (i) any of amino acids K, S, L, Y, G, and P at amino acid position 11 of SEQ ID NO: 4 and/or (ii) any of amino acids T, D, E, and V at amino acid position 12 of SEQ ID NO: 4; optionally wherein the AAV capsid protein comprises SEQ ID NO: 4.

6. The engineered AAV capsid protein of claim 1, comprising a variant sequence of SEQ ID NO: 5, wherein the variant sequence comprises (i) any of amino acids T, M, D, E, Q, S, V, and G at amino acid position 4 of SEQ ID NO: 5 and/or (ii) any of amino acids P, H, R, A, and V at amino acid position 7 of SEQ ID NO: 5; optionally wherein the AAV capsid protein comprises SEQ ID NO: 5.

7. The engineered AAV capsid protein of claim 1, comprising a variant sequence of SEQ ID NO: 6, wherein the variant sequence comprises (i) any of amino acids L, E, R, and V at amino acid position 3 of SEQ ID NO: 6 and/or (ii) any of amino acids T, H, E, A, and P at amino acid position 4 of SEQ ID NO: 6; optionally wherein the AAV capsid protein comprises SEQ ID NO: 6.

8. The engineered capsid protein of claim 1, comprising an amino acid sequence set forth in SEQ ID NO: 1

9. The engineered capsid protein of claim 1, comprising an amino acid sequence set forth in SEQ ID NO: 2.

10. The engineered capsid protein of claim 1, comprising an amino acid sequence set forth in SEQ ID NO: 3.

11. The engineered capsid protein of claim 1, comprising an amino acid sequence set forth in SEQ ID NO: 4.

12. The engineered capsid protein of claim 1, comprising an amino acid sequence set forth in SEQ ID NO: 5.

13. The engineered capsid protein of claim 1, comprising an amino acid sequence set forth in SEQ ID NO: 6.

14. An engineered AAV capsid protein, wherein the engineered AAV capsid protein comprises a variant sequence that is at least 80% identical to SEQ ID NO: 8, optionally wherein there is a substitution of one or two amino acids at any one or two amino acid positions 585, 586, 587, 588, 589, 590, 591, 592, 593, 594, 595, 596, and 597 of SEQ ID NO: 8.

15. The engineered AAV capsid protein of claim 14, wherein the engineered AAV capsid protein comprises a variant sequence that is at least 80% identical to SEQ ID NO: 9.

16. The engineered AAV capsid protein of claim 14, wherein the engineered AAV capsid protein comprises a variant sequence that is at least 80% identical to SEQ ID NO: 10.

17. The engineered AAV capsid protein of claim 14, wherein the engineered AAV capsid protein comprises a variant sequence that is at least 80% identical to SEQ ID NO: 11.

18. The engineered AAV capsid protein of claim 14, wherein the engineered AAV capsid protein comprises a variant sequence that is at least 80% identical to SEQ ID NO: 12.

19. The engineered AAV capsid protein of claim 14, wherein the engineered AAV capsid protein comprises a variant sequence that is at least 80% identical to SEQ ID NO: 13.

20. The engineered AAV capsid protein of claim 14, wherein the engineered AAV capsid protein comprises a variant sequence that is at least 80% identical to SEQ ID NO: 14.


SEQUENCE LISTING (14 sequences)

SEQ ID NO: 1 - protein, 13 aa, synthetic construct
  REGION 1..13: note = Description of sequence: Adeno-associated virus sequence: Variant A
  VARIANT 5: note = Any of lysine, methionine, asparagine, tyrosine, proline
  VARIANT 8: note = Any of methionine, histine, asparagine, serine, threonine, alanine, isoleucine, leucine, phenylalanine, tyrosine, proline
RGNMKLTMQERQA

SEQ ID NO: 2 - protein, 13 aa, synthetic construct
  REGION 1..13: note = Description of sequence: Adeno-associated virus sequence: Variant B
  VARIANT 7: note = Any of lysine, methionine, asparagine, leucine
  VARIANT 8: note = Any of serine, glutamine, methionine
RGNMTLKSQERQA

SEQ ID NO: 3 - protein, 13 aa, synthetic construct
  REGION 1..13: note = Description of sequence: Adeno-associated virus sequence: Variant C
  VARIANT 4: note = Any of lysine, arginine, phenylalanine, tyrosine, glycine
  VARIANT 9: note = Any of Aspartic acid, lysine, asparagine, serine
RGNKTLTRDERQA

SEQ ID NO: 4 - protein, 13 aa, synthetic construct
  REGION 1..13: note = Description of sequence: Adeno-associated virus sequence: Variant D
  VARIANT 11: note = Any of lysine, serine, leucine, tyrosine, glycine, proline
  VARIANT 12: note = Any of threonine, aspartic acid, glutamic acid, valine
RGNMTLTRQEKTA

SEQ ID NO: 5 - protein, 13 aa, synthetic construct
  REGION 1..13: note = Description of sequence: Adeno-associated virus sequence: Variant E
  VARIANT 4: note = Any of threonine, methionine, Aspartic acid, glutamic acid, glutamine, serine, valine, glycine
  VARIANT 7: note = Any of proline, histine, arginine, alanine, valine
RGNTTLPRQERQA

SEQ ID NO: 6 - protein, 13 aa, synthetic construct
  REGION 1..13: note = Description of sequence: Adeno-associated virus sequence: Variant F
  VARIANT 3: note = Any of leucine, glutamic acid, arginine, valine
  VARIANT 4: note = Any of threonine, histine, glutamic acid, alanine, proline
RGLTTLTRQERQA

SEQ ID NO: 7 - protein, 13 aa, synthetic construct
  REGION 1..13: note = Description of sequence: Diversified Region in STAC-102
RGNMTLTRQERQA

SEQ ID NO: 8 - protein, 742 aa, synthetic construct
  REGION 1..742: note = Description of sequence: Modified STAC-102 Parent Capsid
  VARIANT 585..597: note = Position can be any known amino acid
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNMTLTRQERQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - protein, 742 aa, synthetic construct
  REGION 1..742: note = Description of sequence: Parent Capsid STAC-102 with SEQ ID NO: 1, Variant A
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNMKLTMQERQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 10 - protein, 742 aa, synthetic construct
  REGION 1..742: note = Description of sequence: Parent Capsid STAC-102 with SEQ ID NO: 2, Variant B
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNMTLKSQERQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 11 - protein, 742 aa, synthetic construct
  REGION 1..742: note = Description of sequence: Parent Capsid STAC-102 with SEQ ID NO: 3, Variant C
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNKTLTRDERQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 12 - protein, 742 aa, synthetic construct
  REGION 1..742: note = Description of sequence: Parent Capsid STAC-102 with SEQ ID NO: 4, Variant D
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNMTLTRQEKTAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 13 - protein, 742 aa, synthetic construct
  REGION 1..742: note = Description of sequence: Parent Capsid STAC-102 with SEQ ID NO: 5, Variant E
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNTTLPRQERQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 14 - protein, 742 aa, synthetic construct
  REGION 1..742: note = Description of sequence: Parent Capsid STAC-102 with SEQ ID NO: 6, Variant F
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLTTLTRQERQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL